SEQUENCE LISTING (38 sequences)

SEQ ID NO: 1 - DNA, 23 bp, Artificial Sequence
  target region sequence
ctgaacatcatccacgtcactgg

SEQ ID NO: 2 - DNA, 60 bp, Artificial Sequence
  targeted region of WT
aggtggaggacttggaccggctgaacatcatccacgtcactgggacgaaggggaaggtga

SEQ ID NO: 3 - DNA, 55 bp, Artificial Sequence
  targeted region of FPGSko-1
aggtggaggacttggaccggctgaacatcatccactgggacgaaggggaaggtga

SEQ ID NO: 4 - DNA, 53 bp, Artificial Sequence
  targeted region of FPGSko-2
aggtggaggacttggaccggctgaacatctcactgggacgaaggggaaggtga

SEQ ID NO: 5 - DNA, 19 bp, Artificial Sequence
  FPGS GT-F primer
cactggtctgctggctgtc

SEQ ID NO: 6 - DNA, 26 bp, Artificial Sequence
  FPGS GT-R primer
ccaatatggtaagtgctaactgaatg

SEQ ID NO: 7 - DNA, 21 bp, Artificial Sequence
  FPGS GT-F1 primer
gtgaccctcagacacagttgg

SEQ ID NO: 8 - DNA, 22 bp, Artificial Sequence
  FPGS GT-R1 primer
ctaaagaatcccgtcttcaggc

SEQ ID NO: 9 - DNA, 19 bp, Artificial Sequence
  FPGS sqRT-F primer
catgctcaataccctgcag

SEQ ID NO: 10 - DNA, 21 bp, Artificial Sequence
  FPGS sqRT-R primer
cttggtgaagagctcaggact

SEQ ID NO: 11 - DNA, 19 bp, Artificial Sequence
  FPGS qRT-F primer
tggagtaccaggatgccgt

SEQ ID NO: 12 - DNA, 18 bp, Artificial Sequence
  FPGS qRT-R primer
cacaggtggagcccttcc

SEQ ID NO: 13 - DNA, 18 bp, Artificial Sequence
  FPGS WF-F primer
atgtcgcgggcgcggagc

SEQ ID NO: 14 - DNA, 18 bp, Artificial Sequence
  FPGS WF-R primer
ctgggacagtgcgggctc

SEQ ID NO: 15 - DNA, 17 bp, Artificial Sequence
  bActin forward primer
cttccttcctgggcatg

SEQ ID NO: 16 - DNA, 24 bp, Artificial Sequence
  bActin reverse primer
gagcaatgatcttgatcttcattg

SEQ ID NO: 17 - DNA, 23 bp, Artificial Sequence
  GTSF1 forward primer
gtgcagaaagaatcatcctgatg

SEQ ID NO: 18 - DNA, 24 bp, Artificial Sequence
  GTSF1 reverse primer
ccacaaatctttatcccagtcttc

SEQ ID NO: 19 - DNA, 25 bp, Artificial Sequence
  SLC7A11 forward primer
ctatttggagctttgtcttatgctg

SEQ ID NO: 20 - DNA, 22 bp, Artificial Sequence
  SLC7A11 reverse primer
cactacagttatgcccacagct

SEQ ID NO: 21 - DNA, 21 bp, Artificial Sequence
  ANOS1 forward primer
gcttttgtgagcctctcttcc

SEQ ID NO: 22 - DNA, 24 bp, Artificial Sequence
  ANOS1 reverse primer
gggacacctttgtacagagtcttg

SEQ ID NO: 23 - DNA, 27 bp, Artificial Sequence
  GABRB2 forward primer
agtcaatatggattataccttgacaat

SEQ ID NO: 24 - DNA, 24 bp, Artificial Sequence
  GABRB2 reverse primer
ggttgtgattctgagtccataaag

SEQ ID NO: 25 - DNA, 23 bp, Artificial Sequence
  ANKRD1 forward primer
gtagaggaactggtcactggaaa

SEQ ID NO: 26 - DNA, 19 bp, Artificial Sequence
  ANKRD1 reverse primer
ttgagctctgcctctcgtt

SEQ ID NO: 27 - DNA, 19 bp, Artificial Sequence
  DKK1 forward primer
caactaccagccgtacccg

SEQ ID NO: 28 - DNA, 23 bp, Artificial Sequence
  DKK1 reverse primer
cacacatattccatttttgcagt

SEQ ID NO: 29 - DNA, 23 bp, Artificial Sequence
  MTHFD2 forward primer
ggcagttcgaaatgaagctgttg

SEQ ID NO: 30 - DNA, 22 bp, Artificial Sequence
  MTHFD2 reverse primer
ctgttgattcccacaactgcag

SEQ ID NO: 31 - DNA, 19 bp, Artificial Sequence
  ALDH1L2 forward primer
cactggccgggtttatttc

SEQ ID NO: 32 - DNA, 18 bp, Artificial Sequence
  ALDH1L2 reverse primer
ctgcagccaaagccagag

SEQ ID NO: 33 - DNA, 23 bp, Artificial Sequence
  Nanog forward primer
cagctacaaacaggtgaagacct

SEQ ID NO: 34 - DNA, 18 bp, Artificial Sequence
  Nanog reverse primer
ggttcaccaggcatccct

SEQ ID NO: 35 - DNA, 20 bp, Artificial Sequence
  Sox2 forward primer
catgaatgccttcatggtgt

SEQ ID NO: 36 - DNA, 19 bp, Artificial Sequence
  Sox2 reverse primer
gtgctccttcatgtgcagc

SEQ ID NO: 37 - DNA, 19 bp, Artificial Sequence
  Oct4 forward primer
cggaggagtcccaggacat

SEQ ID NO: 38 - DNA, 24 bp, Artificial Sequence
  Oct4 reverse primer
ctgaataccttcccaaatagaacc

(19) United States
(12) Patent Application Publication
(10) Pub. No.: US 2021/0214692 A1
(43) Pub. Date: Jul. 15, 2021

(54) METHODS OF REPROGRAMMING SOMATIC CELLS AND MATERIALS RELATED THERETO

(71) Applicant: CITY OF HOPE, Duarte, CA (US)

(72) Inventors: Arthur D. RIGGS, Duarte, CA (US); Avinash C. SRIVASTAVA, Duarte, CA (US); Timothy R. O'CONNOR, Duarte, CA (US)

(73) Assignee: CITY OF HOPE, Duarte, CA (US)

(21) Appl. No.: 16/950,751

(22) Filed: Nov. 17, 2020

Related U.S. Application Data
(60) Provisional application No. 62/937,940, filed on Nov. 20, 2019.

(51) Int. Cl. C12N 5/074

(52) U.S. Cl. CPC C12N 5/0696; C12N 2501/84; C12N 2501/999

(57) ABSTRACT

Disclosed herein are methods for reprogramming a somatic cell into a pluripotent stem cell by contacting the somatic cell with one or more antifolate agents, with or without methionine, in vitro for a period of time sufficient to reprogramming the somatic cell and selecting and growing the cells that express one or more stem cell markers. Also disclosed are induced pluripotent stem cells obtained from somatic cells.

### PRIORITY CLAIM

This application claims priority to U.S. Provisional Application No. 62/937,940, filed Nov. 20, 2019, which is incorporated by reference herein in its entirety, including drawings.

### SEQUENCE LISTING

This application contains a Sequence Listing, which was submitted in ASCII format via EFSWeb, and is hereby incorporated by reference in its entirety. The ASCII copy, created on Mar. 8, 2021, is named SequenceListing.txt and is 8 KB in size.

### BACKGROUND

A stem cell can naturally divide or differentiate into another stem cell, progenitor, precursor, or somatic cell. However, sometimes a transient change in somatic cells can change its phenotype or express certain markers when placed in certain conditions. In this situation, the phenotype of many cells can be changed through forced expression of certain genes. However, once these factors are removed, the cells revert back to their original state. Therefore, these mechanisms of cell reprogramming have limited use.

True reprogramming was achieved with induced pluripotent stem cells (iPS cells or iPSCs) created independently by Yamanaka's group (71) and Thomson's group (72), although many of these cells were later found to be cancerous. These cells can be induced by true reprogramming since it was later shown that they can also be induced by non-gene integrating transient transfection (95, 96), by RNA (97) or protein (16, 98) or by small molecules (99). However, these cells are essentially identical to embryonic stem cells and have the same problems of uncontrolled growth, teratoma formation, and potential tumor formation.

Ideally, multipotent stem cells or pluripotent-like cells whose lineage and differentiation potential are more restricted can be developed so that they do not readily form teratomas or have uncontrolled growth. Thus, there is a need for methods of creating multipotent stem cells, multipotent stem-like cells, and stem-like cells and method of reprogramming or transforming easily obtainable cells to highly desirable multipotent stem cells, multipotent stem-like cells, and stem-like cells. This disclosure provides methods and materials for reprograming an easily obtainable cell into a cell that is generally difficult to obtain, or reprograming a vegetal cell to have new or different functionalities, without using stem cells.

### SUMMARY

In one aspect, provided is a method of reprogramming somatic cells into pluripotent stem cells. The method includes contacting one or more somatic cells with one or more antifolate agents in vitro for a period of time sufficient to induce reprogramming of the somatic cells, selecting one or more cells expressing one or more stem cell markers, growing the selected cells in a suitable medium to obtain the induced pluripotent stem cells (iPSCs). In certain embodiments, the method further comprises contacting the one or more somatic cells with one or more of glutamine, glutamate, arginine, methionine, and GABA. The method can further include growing the iPSCs in a suitable differentiation medium such that the selected cells differentiate into a desired lineage. In certain embodiments, the somatic cell includes but is not limited to a fibroblast cell and a stromal cell. In certain embodiments, the antifolate agent is a natural compound or a synthetic compound. In certain embodiments, the antifolate agent inhibits one or more C1 metabolites. In certain embodiments, the antifolate agent inhibits thymidylate synthase (TS), dihydrofolate reductase (DHFR), or both. In certain embodiments, the antifolate agent inhibits folypolyglutamate synthetase (FPGS). In certain embodiments, the antifolate agent includes but is not limited to methotrexate (MTX), pemetrexed (PTX), aminopterin (AMT), raltitrexed, trimetrexate, piritrexim, edatrexate, and fluorouracil. In certain embodiments, the induced pluripotent stem cell expresses one or more of the stem cell markers including OCT4, SOX2, SSEA-4, Nanog, and TRA 1-60. In certain embodiments, the somatic cell is contacted with the antifolate agent for at least 1 day, at least 2 days, at least 3 days, at least 4 days, at least 5 days, at least 6 days, at least 7 days, such as 7 days, 8 days, 9 days, 10 days, 11 days, 12 days, 13 days, 14 days, or 15 days.

In another aspect, provided is an induced pluripotent stem cell (iPSC) obtained by reprogramming a somatic cell including but not limited to a fibroblast cell and a stromal cell. The obtained pluripotent stem cell expresses one or more of the stem cell markers including OCT4, SOX2, SSEA-4, Nanog, and TRA 1-60. In certain embodiments, the obtained pluripotent stem cell is capable of differentiating into 3 germ layers including mesoderm, endoderm, and ectoderm. In certain embodiments, the obtained pluripotent stem cell is capable of differentiating into a cardiomyocyte or a neuron. The reprogramming method includes contacting one or more somatic cells with one or more antifolate agents in vitro for a period of time sufficient to induce reprogramming of the somatic cell, selecting one or more cells expressing one or more stem cell markers, growing the selected cells in a suitable medium to obtain the induced pluripotent stem cells (iPSCs). In certain embodiments, the method further comprises contacting the one or more somatic cells with one or more of glutamine, glutamate, arginine, methionine, and GABA. The method can further include growing the iPSCs in a suitable differentiation medium such that the selected cells differentiate into a desired lineage. In certain embodiments, the antifolate agent is a natural compound or a synthetic compound. In certain embodiments, the antifolate agent inhibits one or more C1 metabolites. In certain embodiments, the antifolate agent inhibits thymidylate synthase (TS), dihydrofolate reductase (DHFR), or both. In certain embodiments, the antifolate agent inhibits folypolyglutamate synthetase (FPGS). In certain embodiments, the antifolate agent includes but is not limited to methotrexate (MTX), pemetrexed (PTX), aminopterin (AMT), raltitrexed, trimetrexate, piritrexim, edatrexate, and fluorouracil. In certain embodiments, the somatic cell is contacted with the antifolate agent for at least 1 day, at least 2 days, at least 3 days, at least 4 days, at least 5 days, at least 6 days, at least 7 days, such as 7 days, 8 days, 9 days, 10 days, 11 days, 12 days, 13 days, 14 days, or 15 days.

### BRIEF DESCRIPTION OF THE DRAWINGS

This application contains at least one drawing executed in color. Copies of this application with color drawing(s) will be provided by the Office upon request and payment of the necessary fees.

FIG. 1 is a schematic representation of THF production and C1 metabolism and their distribution in different compartments of the mammalian cell [adapted from Tibbetts and Appling (1) and Ducker and Rabinowitz (94)]. Activated C1 units, monoglutamylated THFs, are transported from cytoplasm to mitochondria where they are polyglutamylated by FPGS, and polyglutamated folates are utilized in C1 metabolism by SHMT. MTHFD1 is trifunctional in the cytoplasm. MTHFD1L is monofunctional, and MTHFD2 or MTHFD2L are bifunctional in mitochondria. 10-formyl-THF dehydrogenase is functional in both compartments in mammals. All abbreviations are standard gene names. Certain descriptions utilize the common protein name for clarity. ALDH1L1, cytosolic 10-formyl-THF dehydrogenase; ALDH1L2, mitochondrial 10-formyl-THF dehydrogenase; ATIC, 5-am inoimidazole-4-carboxam ide ribonucleotide formyltransferase/IMP cyclohydrolase; DHFR, dihydrofolate reductase; dTMP, thymidine monophosphate; GART, phosphoribosylglycinamide formyltransferase; Hcy, homocysteine; MTFMT, mitochondrial methionyl-tRNA formyltransferase; MTHFD1, MTHFD, cyclohydrolase, and formyl-THF synthetase 1; MTHFD1L, monofunctional THF synthase (mitochondrial); MTHFD2L, MTHFD2-like; MTHFR, methylene THF reductase; MTR, methionine synthase; SHMT1, cytosolic SHMT; SHMT2, mitochondrial SHMT; THF-Glut tetrahydrofolate monoglutamate; THF-Glun, tetrahydrofolate polyglutamate; TYMS, thymidylate synthetase.

FIGS. 2A-2C show FPGS knock out from 293T cells. FIG. 2A shows the CRISPR/Cas9 construct to knock out mitochondrial and cytoplasmic isoforms of FPGS from 293T (SEQ ID NO:1). FIGS. 2B-2C show semi-quantitative RT-PCR and qRT-PCR of FPGS^{ko} cell lines showing less or no transcripts in the mutant. FIG. 2B shows semi-quantitative RT-PCR (25-PCR cycles) of FPGS^{ko} and WT (293T), and FIG. 2C shows qRT-PCR of FPGS^{ko}-1 and FPGS^{ko}-2 showed no FPGS transcripts compare to WT cell lines suggesting either expression is quite low or no detectable FPGS transcripts in the cell line. β-actin was used as a control. The asterisk indicates a statistically significant difference according to Student's t-test (*P<0.05).

FIGS. 3A-3C show generation of FPGS^{ko} 293T cell lines using CRISPR/Cas9. FIG. 3A shows a schematic representation of the FPGS exons (E1-E15), with exon 4 indicated. PAM, protospacer-adjacent motif. FIG. 3B shows the sequences of the targeted regions of WT (SEQ ID NO:2) and FPGS^{ko}-1 (SEQ ID NO:3) and FPGS^{ko}-2 (SEQ ID NO:4) cell lines. FIG. 3C are Western blots showing loss of FPGS in FPGS^{ko} lines (C1). Probing the membrane for β-actin showed that sample loading for all 3 samples was similar (C2). The arrows indicate the FPGS or actin positions, as well as that of a nonspecific band that appears.

FIGS. 4A-4B show that FPGS deletion decreases cell proliferation and changes cellular morphology. FIG. 4A shows that adherent cell growth of FPGS, FPGS^{ko}-1, and FPGS^{ko}-2 was assessed by cell counting at the indicated times. A total of 4350 cells were seeded in 12-well plates, and cells were counted after 8 days. The number of cells for each FPGS^{ko} cell lines (FPGS^{ko}-1 and FPGS^{ko}-2) were compared with the parental 293T cells. FIG. 4B shows the genetic complementation of FPGS^{ko}-1 and FPGS^{ko}-2 mutants. Transfection of an FPGS expression plasmid into FPGS^{ko}-1 and FPGS^{ko}-2 rescued the phenotype (Complemented FPGS^{ko} Comp-FPGS^{ko}-1 and Comp-FPGS^{ko}-2). Error bars indicate means±SE (n=5). ***P<0.0001.

FIGS. 5A-5B show genetic complementation of FPGS^{ko}-1 and FPGS^{ko}-2 mutants. FIG. 5A shows that a functional FPGS transfected to FPGS^{ko}-1 and FPGS^{ko}-2 mutants rescued the phenotype. FIG. 5B shows that a semi-quantitative PCR (28 cycle) and qRT-PCR confirmed the presence of FPGS transcripts in the complemented lines.

FIG. 6 shows the quiescent energy phenotype of the FPGS^{ko}-1. An Agilent Seahorse XF was used to determine OCR and ECAR of FPGS^{ko}-1 and 293T. *P<0.05, **P<0.001 (Student's t test).

FIGS. 7A-7D show the quantitative estimation of SAM, SAH, Gln, Glu, and GABA by HILIC and metabolomic profiling by GC-MS. Quantitative estimation of SAM and SAH (FIG. 7A) was determined using HILIC. Metabolic profiling of amino acids (FIG. 7B) depicting fold change (logarithmic values) in the FPGS^{ko} cell line compared with the parental 293T cells, which was confirmed by the quantitative estimation of Gln, Glu, and GABA by HILIC (FIG. 7C). Metabolic profiling of nucleic acids (FIG. 7D) depicting fold change (logarithmic values) in the FPGS^{ko} cell line compared with the parental 293T cells. AMOT, angiomotin; CDR1, cerebellar degeneration related protein 1; CHAC1, γ-glutamylcyclotransferase 1; CNPY1, canopy FGF signaling regulator 1; CSMD3, CUB and Sushi multiple domains 3; DDR2, discoidin domain-containing receptor 2; DPYD, dihydropyrimidine dehydrogenase; DUSP6, dual specificity phosphatase 6; ETV5, ETS variant 5; GABRA3, GABA type A receptor a3 subunit; GABRB2, GABA type A receptor b2 subunit; GABRB3, GABA type A receptor b3 subunit; GDPD3, glycerophosphodiester phosphodiesterase domain containing 3; HSPB8, heat shock protein family B member 8; IRS4, insulin receptor substrate 4; KRTAP21-2, keratin-associated protein 21-2; LCP1, lymphocyte cytosolic protein 1; MAP3K12, MAPKK kinase 12; NEFM, neurofilament medium; PSAT1, phosphoserine aminotransferase 1; RHEBL1, Ras homolog enriched in brain like 1; SERPINF1, serpin family F member 1; SLC6A9, solute carrier family 6 member 9; TXNIP, thoredoxin-interacting protein. Error bars represent the SE for 5 independent experiments and 5 technical replicates. *P<0.05, **P<0.01, ***P<0.001 (Student's t test).

FIGS. 8A-8C show differentially expressed genes of FPGS^{ko} cell line. FIG. 8A shows scatter plot transcription signals determined by microarray analysis of FPGS^{ko}-1 and 293T. FIG. 8B shows a summary of the number of differentially expressed genes. FIG. 8C shows the heat map of the most differentially expressed genes. Avg, average.

FIG. 9 shows the validation of microarray gene expression data by qRT-PCR. Relative expression levels of GTSF1, SLC7A11, ALDH1L2, MTHFD2, ANOS1, GABA receptor subunit β-2 (GABRB2), ANKRD1, and DKK1 genes in FPGS^{ko} (mutant) and FPGS (control-293T) cells were checked to validate microarray expression data. Error bars represent the SE for 3 independent experiments and 3 technical replicates. *P<0.05, Student's t test. *P<0.05, **P<0.001 (Student's t test).

FIGS. 10A-C: 10A shows that FPGS mutants showed a significant reduction in global DNA methylation. 5-mC content in the FPGS^{ko} cell line was measured and compared with the parental 293T cells. DNA was extracted and equal amounts of genomic DNA (100 ng) were analyzed with 5-mC ELISA. Statistical analysis was performed using Student's t test. *P<0.05 indicates significantly lower levels of DNA methylation in the mutant in comparison with controls. Error bars represent the SE for at least 3 independent experiments and 3 technical replicates. FIGS. 10B and 10C show the relative expression levels of Oct4 and Sox2 genes (FIG. 10B) and immunofluorescence localization of SSEA4 and Oct4 in FPGS^{ko} and control (293T) cells to evaluate pluripotency biomarkers (FIG. 10C). The FPGS^{ko} exhibits different cell morphology compared with WT cells (FIG. 10C). The FPGS^{ko} illustrates the expression of SSEA4 surface antigens but WT had no signal (FIG. 10C). Immunoreactivity for the OCT4 transcription factor in the mutant was found in the nucleus and cytoplasm; however, no signals were detected in WT (FIG. 10C). DAPI staining in FPGS^{ko} and WT were used to validate live cells. Scale bar, 200 mm. Error bars represent the SE for 3 independent experiments and 3 technical replicates. *P<0.05, **P<0.001 (Student's t test).

FIG. 11 shows the relative transcripts of NKX2, MYL2, and cTNT gene in FPGS^{ko} and WT (293T) cells to check the expression of cardiac markers. Nkx2 (an early cardiac transcriptional factor, indicative of cardiac progenitor phenotype), MYL2 (Myosin regulatory light chain 2, a distinctly expressed protein in cardiac muscle), and cTNT (cardiac troponin T, a muscle contractility regulatory protein, indicative of a mature cardiac phenotype) were significantly up-regulated in the FPGS^{ko} cells grown in basal 10% FBS/DMEM medium. The values were normalized against β-actin as housekeeping gene. Error bars represent the standard error for three independent experiments and three technical replicates. The asterisk indicates a statistically significant difference according to Student's t-test (*P<0.05; **P<0.01).

FIG. 12 shows the phenotypic description of the FPGS^{ko} on differentiation medium (RPMI1640+1327). Comparative analysis showed prominent neurogenesis in the mutant and close analysis of the neurons show that these could be bipolar neurons.

FIGS. 13A-13B show the relative transcripts of glutamate-ammonia ligase (FIG. 13A) and free glutamate concentration (FIG. 13B) in FPGS^{ko} and WT (293T) cells: The expression of GLUL (glutamate-ammonia ligase) was significantly low and free glutamate concentration was significantly high in the mutant. The values were normalized against β-actin as housekeeping gene. Error bars represent the standard error for three independent experiments and three technical replicates. The asterisk indicates a statistically significant difference according to Student's t-test (*P<0.05; **P<0.01).

FIG. 14 shows that supplementation with thymidine, 5-CHO-THF, and amino acids complemented the phenotype of FPGS^{ko}. The growth medium was supplemented with 1 and 2× essential amino acids (EAAs), and cells were grown for 7 days (A2-A3). Additionally, to check the growth behavior of the mutant in a different basal medium, regular DMEM and IMDM with 1×NEAAs were tested. To rescue the phenotype of FPGS^{ko}, 5-CHO-THF (1 mM) with sodium hypoxanthine (10 mM) and thymidine (1.6 mM) mixture (HT; Thermo Fisher Scientific) was exogenously applied to IMDM, respectively (A5, A6), and compared with the FPGS^{ko} cells (A1) and WT cells (A6) grown with only solvent as a control. All modifications showed improved growth of FPGS^{ko}-1, ranging from partial (A2-A5) to full (A6) complementation. All the experiments were carried out in triplicate and cell proliferation was measured using a Cellometer. The number of cells for each cell line is compared with the number of colonies for parental 293T cells. Error bars indicate means±SE (n=4). *P<0.05, **P<0.001.

FIG. 15 shows a schematic model to illustrate role of FPGS and connected pathways in DNA methylation and pluripotency. Cells with functional FPGS (WT) rely on appropriate production and assimilation of Gln-Glu-GABA in a cyclic manner through the tricarboxylic acid (TCA) cycle (left); nonfunctional FPGS)(FPGS^{ko}) perturbs the Gln-Glu-GABA equilibrium and promotes cardio- and neurogenesis utilizing excess GABA in the system (right). Black arrows represent normal enzymatic reactions in the cycle; red arrows indicate possible consequences caused by FPGS deletion. Hcy, homocysteine; Met, methionine.

FIGS. 16A-16B show that prolong, controlled application of MTX and PTX resulted in reduced cell proliferation in human embryonic kidney cells (293T), human fibroblast cells (HF57), and human dermal fibroblast (HDF) cells. FIG. 16A shows HEK293T cells treated with MTX and PTX at the indicated concentrations, and FIG. 16B shows treatment of all three cell lines, 293T, HF57, and HDF with MTX and PTC, with or without HT. The cells were grown in 5% dialyzed-FBS DMEM medium. 293T cells were treated with 500 nM MTX, and HF57 cells and HDF cells were treated with 1 μM MTX for 7-days. Because inhibition of DHFR and TS by folate antagonists (MTX, PTX) results in a deficiency in the cellular pools of thymidylate and purines and thus a decrease in nucleic acid synthesis, sodium hypoxanthine (10 mM) and thymidine (1.6 mM) were added to the medium to overcome the effects of MTX and PTX. The addition of sodium hypoxanthine and thymidine (HT) showed almost full restoration of growth rate of MTX and PTX treated HEK293T, HF57 and HDF cell lines. The cells were harvested for DNA/RNA isolation and analyzed for gene expression and DNA methylation.

FIG. 17 shows that the HEK293T cells were sensitive to PTX and MTX and only 20-25% cells were viable after 5-days of MTX or PTX treatment. However, both fibroblast cell lines (HF57 and HDF) demonstrated better cell viability (almost 60-75% viability post MTX or PTX treatment) compared to 293T cells.

FIG. 18 shows a significant reduction in global DNA methylation in MTX and PTX treated 293T and fibroblast cells: 5-methylcytosine (5mC) content in MTX and PTX treated (7-days) 293T and HF57 cell lines were measured and compared with the untreated cells. Equal amount of DNA (100 ng) was analyzed with 5mC enzyme-linked immunosorbent assay (ELISA) (EpiGentek). Statistical analysis was performed using Student's t-test. **p<0.001 indicates significantly lower levels of DNA methylation in the mutant in comparison to controls. Error bars represent the standard error for at least three independent experiments and three technical replicates.

FIG. 19 shows a significant reduction in histone methylation levels in HEK293T and HF57 cells. Dimethyl H3-K9 and global histone H3-K4 methylation in MTX and PTX treated 293T and HF57 cells were significantly reduced. Calculation of methylation level (based on OD values as shown on y-axis) was performed according to manufacturer's instructions. Representative histogram from three independent experiments are shown. All data are presented as the mean±standard error (SE) of triplicate measurements. *P<0.05, ***P<0.001.

FIGS. 20A-D: FIG. 20A shows transcriptomics of MTX and PTX treated HF57 cells. Close examination of one carbon-metabolism and DNA methylation related genes showed that about 41 genes were significantly down-regulated and 9 genes were upregulated in the MTX and PTX treated cells. RNAseq data validated that application of MTX and PTX affected C1 metabolism in the treated cells. Compared to controls, about 50 pluripotency markers were significantly enriched in MTX or PTX treated cell including NANOG (6-fold), LIN28A (6-fold), and SOX2 (5-fold) indicating changed cell plasticity. The global transcriptional change across the groups compared was visualized by a volcano plot (FIGS. 20A-20B). Each data point in the scatter plot represents a gene. The log 2 fold change of each gene is represented on the x-axis and the log 10 of its adjusted p-value is on the y-axis. Genes with an adjusted p-value less than 0.05 and a log 2 fold change greater than 1 are indicated by red dots. These represent up-regulated genes. Genes with an adjusted p-value less than 0.05 and a log 2 fold change less than −1 are indicated by green dots. These represent down-regulated genes. Heat map visualization of the selected 67 differentially expressed genes. Original values are ln(x)-transformed. Rows are centered; unit variance scaling is applied to rows. Red indicates higher expression, whereas low expression indicated in green (FIG. 20C). For visual clarity, expression of selected genes is presented in groups based on their relative values (FIG. 20D).

FIGS. 21A-B: FIG. 21A shows that the relative transcripts levels of Sox2 and Oct4 (stem cell markers) were significantly higher in MTX treated cells compared to control cells. FIG. 21B shows that the relative transcripts levels of Oct4, Sox2, and Nanog (stem cell markers) were significantly higher in MTX and PTX treated HF57 cells compared to control cells. Also, transcripts levels of MTHFR, MTR and DNMT1 were significantly reduced in MTX and PTX treated HF57 cells.

FIG. 22 shows that the relative transcripts levels of Ankyrin repeat domain 1 (cardiac muscle) and ANOS were significantly higher in MTX treated 293T cells compared to control cells.

FIG. 23 shows that the expression of ANKRD1 and ANOS was also significantly higher in PTX treated 293T cells compared to control cells.

FIGS. 24A-24B show that the human fibroblast (HF57) cells were treated with 1 μM MTX and 1 μM PTX for 12-days. Post MTX or PTX treatment, the cells were fixed to examine the stem cell markers. The cells were grown in regular 5% FBS and 10% FBS in DMEM medium to check the difference. Scale Bar=400 μM (FIG. 24A); Scale Bar=200 μM (FIG. 24B).

FIG. 25 shows that the human fibroblast (HF57) cells were treated with 1 μM MTX for 9-days. Post MTX treatment, the cells were labeled using primary antibodies (ant-SSEA4-anti mouse IgG3) followed by secondary antibodies conjugated to Alexa Fluor 488 goat anti-mouse IgG3. The cells were counterstained with DAPI. MTX treated cells illustrate the expression of SSEA-4 surface antigens, however, no signals were detected in control cells. The cells were grown in regular DMEM medium with or without MTX. Scale Bar=400 μM.

FIGS. 26A-26B show the expression of SSEA-4 and TRA 1-60 surface markers in MTX and PTX treated HF57 cells. The human fibroblast (HF57) cells were treated with 1 μM MTX for 7 days. Post MTX treatment, the cells were labeled using primary antibodies (anti-SSEA4-anti mouse IgG3 and anti-TRA 1-60 host-rabbit) followed by secondary antibodies conjugated to (Alexa Fluor 488 goat anti-mouse IgG3 and Alexa Fluor 594 donkey anti-rabbit). The cells were counterstained with DAPI. MTX treated cells illustrate the expression of SSEA-4 and TRA 1-60 surface antigens. The cells were grown in DMEM medium with low methionine (15 mg/L). Scale Bar=400 μM (FIG. 26A); Scale Bar=200 μM (FIG. 26B).

FIGS. 27A-B: FIG. 27A shows that MTX or PTX treated 293T cells formed embryoid bodies. FIG. 27B shows the putative pluripotent embryonic bodies derived from 293T cells treated with MTX and PTX. HEK293T and HF57 were treated with MTX and PTX for 7 or more days in low methionine condition. SSEA4 positive cells were sorted using cell sorter and maintained on mTeSR™ Plus medium supplemented with HT. After 15-days cells start forming embryoid bodies in both the cell lines indicating altered epigenetic and metabolic programing.

FIG. 28 shows three-germ layer immunostaining of putative pluripotent stem cell derived from 293T cells treated with MTX: Alexa Fluor™ 488 goat anti-mouse IgG1; for use with anti-AFP (Green) and Alexa Fluor™ 594 goat anti-mouse IgG2a; for use with anti-SMA (Red).

FIGS. 29A-B: FIG. 29A shows three-germ layer immunostaining of putative pluripotent stem cell derived from 293T cells treated with PTX. This was compared with human embryonic stem cell (H1) and commercial iPSC: Alexa Fluor™ 488 goat anti-mouse IgG1; for use with anti-AFP (Green) and Alexa Fluor™ 594 goat anti-mouse IgG2a; for use with anti-SMA (Red). FIG. 29B shows three-germ layer immunostaining of putative pluripotent stem cell derived from 293T and HF57 cells treated with MTX compared with the induced pluripotent stem cell (iPSCs) and embryonic stem cells (H1-ESCs): Alexa Fluor™ 488 goat anti-mouse IgG1; for use with anti-AFP (Green); Alexa Fluor™ 594 goat anti-mouse IgG2a; for use with anti-SMA (Red), and Alexa Fluor® 488 donkey anti-rabbit; for use with anti-TUJ1. Because absorption spectrum for anti-AFP and anti-TUJ1 were the same, immunostaining of AFP and SMA is shown here.

FIG. 30 shows comparative immunostaining of ESC (H1), iPSC, and MTX and PTX treated 293T and HF57 (Human Foreskin Fibroblast) cells with Nestin (Neural Stem marker) after growing the cells in neural differentiation medium for 12 days.

FIG. 31 shows comparative immunostaining of ESC, iPSc, and putative cardiomyocytes derived from MTX treated HF57 and HEK293T with cardiomyocytes marker (NKX2 and cTNT) after growing the cell in cardiomyocytes differentiation medium for 21 days.

FIG. 32 shows that the HF57 (human fibroblast cells) were treated with PTX for 2-days and expression of ANKRD1 and ANOS was checked. The expression of ANKRD1 and ANOS was significantly higher than control and similar to MTX treated cells. Expression of ANOS was also high in PTX treated cells although some replications showed variable results resulting in high standard deviation. Also, the cells were treated with PTX for 2-days only. Since exposure time of PTX or MTX is very critical for gene expression or other genetic changes, the expression of ANOS could be higher after prolonged exposure to PTX.

### DETAILED DESCRIPTION

Disclosed herein are methods of reprogramming somatic cells into pluripotent stem cells by exposing the somatic cells to a high concentration of one or more antifolate agents such as MTX and PTX in vitro for an extended period of time sufficient for the somatic cells being reprogrammed into pluripotent stem cells, and selecting the pluripotent stem cells expressing one or more of the stem cell markers. In certain embodiments, the somatic cells are treated with variable concentrations of one or more of glutamine, glutamate, arginine, methionine, and GABA in addition to the one or more antifolate agents. In certain embodiments, both the antifolate agent(s) and the one or more of glutamine, glutamate, arginine, methionine, and GABA are added to the medium for growing the somatic cells. For example, the somatic cells are grown in a medium containing one or more of glutamine, glutamate, arginine, methionine, and GABA before and/or after treatment with the antifolate agent(s). In another example, the somatic cells are treated with the antifolate agent(s) while growing in a medium containing one or more of glutamine, glutamate, arginine, methionine, and GABA.

It was expected that at a high concentration, the antifolate agent would kill the cells or at least disrupt cell functions to a certain extent. Surprisingly, the inventors discovered that the treatment with the antifolate agents reprogrammed the somatic cell into an induced pluripotent stem cell rather than killing the somatic cell. The induced pluripotent stem cell expresses a number of stem cell markers, for example, one or more of the markers including OCT4, SOX2, SSEA-4, Nanog, and TRA 1-60, and is capable of differentiating into other types of cells such as cardiomyocytes and neurons.

### Reprogramming Methods

Various concentrations of antifolate agents particularly methotrexate (MTX) and pemetrexed (PTX) were experimented in the cell culture medium. The standard practice to grow the normal/primary/somatic cells is 10% FBS-DMEM medium. As disclosed herein, various types and concentrations of FBS and different media formulations were tested to obtain the effective impact of MTX and/or PTX in less time. In some embodiments, the media formulations contain exogenous methionine at a range of between 0 and 30 mg/L such as about 1 mg/L, about 2 mg/L, about 3 mg/L, about 4 mg/L, about 5 mg/L, about 6 mg/L, about 7 mg/L, about 8 mg/L, about 9 mg/L, about 10 mg/L, about 11 mg/L, about 12 mg/L, about 13 mg/L, about 14 mg/L, about 15 mg/L, about 16 mg/L, about 17 mg/L, about 18 mg/L, about 19 mg/L, about 20 mg/L, about 21 mg/L, about 22 mg/L, about 23 mg/L, about 24 mg/L, about 25 mg/L, about 26 mg/L, about 27 mg/L, about 28 mg/L, about 29 mg/L, or about 30 mg/L, and the somatic cells were grown from 1 to 15 days such as 1 day, 2 days, 3 days, 4 days, 5 days, 6 days, 7 days, 8 days, 9 days, 10 days, 11 days, 12 days, 13 days, 14 days, or 15 days to induce reprogramming of somatic cells into iPSCs. Initially HEK 293T cells were used as the parental cells for the experiment. Once the positive results were obtained with the 293T cell, additional cells such as HF57 (human foreskin fibroblast) and DF (dermal fibroblast) cell lines were used as well.

To induce reprogramming of somatic cells into iPSCs, the parental somatic cells were grown in no methionine, low methionine (7.5 mg/L and 15 mg/L) to regular methionine (30 mg/L) DMEM+5% dialyzed-FBS supplemented with 1% glutamine, sodium hypoxanthine (10 mM) and thymidine (1.6 mM), and then treated for 1-15 days with one or more antifolate agents such as MTX, PTX, or both at various concentrations. For example, 293T cells were treated with 500 nM MTX and/or 1 μM PTX, and HF57 cells and DF cells were treated with 1 μM MTX and/or 1 μM PTX. Immunostaining of live cells with one or more stem cell markers such as SSEA4 (a pluripotentcy surface marker) was performed and the reprogrammed cells positively expressing the stem cell markers were selected using FACS. The independent cell colonies were selected and grown in an FBS-free stem cell medium such as MTeSR medium. These selected cells can be passaged to appropriate differentiation medium and grown into different lineages.

Pluripotent cells can differentiate into all cell types, so these cells can be a biological resource for regenerative medicine. Somatic cells can acquire the embryonic stem cell-like pluripotency by the introduction of four transcription factors, Oct4, Sox2, Klf4 and c-Myc (Yamanaka factors). Murine ES-like cell lines were established from mouse embryonic fibroblasts (MEFs) and skin fibroblasts by simply expressing these four transcription factor genes encoding Oct4, Sox2, Klf4, and c-Myc (71). After introducing active transcription factors into somatic cells, the somatic cells closely resemble ES cells in gene expression patterns, cell biologic and phenotypic characteristics. Since then, numerous studies have established that these transcription factors mediate reprogramming regardless of developmental origins and epigenetic states of a cell. These transcription factors are non-functional in somatic cells due to gene silencing. As demonstrated herein, the expression of Oct4, Sox2, Nanog, and SSEA4 was significantly higher in the MTX/PTX treated cells. Using the technology disclosed herein, nuclear reprogramming of somatic cells was induced possibly due to active Oct4, Sox2, Nanog, and other pluripotency genes, reduced DNA methylation, reduced histone methylation, and altered metabolism of certain biochemical factors (such as glutamine, glutamate, and GABA). This leads to the formation of embryoid bodies from single cells and differentiation into the mesoderm, endoderm, and neuronal ectoderm lineages with high efficiency in the differentiation medium. The 3-Germ Layer Immunocytochemistry Kit (ThermoFisher Scientific, USA), which analyzes spontaneously differentiated embryoid bodies derived from pluripotent stem cells for the presence of all 3-germ layers, was used to confirm the reprogramming. The assay detected widely accepted markers characteristic of the three embryonic germ layers: beta-III tubulin (TUJ1) for ectoderm, smooth muscle actin (SMA) for mesoderm, and alpha-fetoprotein (AFP) for endoderm. Thus, the presence of SMA (mesoderm) and AFP (endoderm) was confirmed using this kit as demonstrated in the working example. The ectoderm which differentiates to form the nervous system (spine, peripheral nerves, neurons, and brain) was not tested with this kit; however, the presence of neurons was confirmed by a different marker. A Human Neural Stem Cell Immunocytochemistry Kit (ThermoFisher Scientific, USA) which enables a convenient image-based analysis of common markers of human neural stem cells such as Nestin was used. The findings clearly show that neural lineages were obtained with high efficiency in the neural differentiation medium. Together, the disclosed methods result in the presence of all 3-germ layers in the MTX/PTX treated cells and these treated cells can differentiate into all cell types including beta cells.

### Antifolate Agents for Inducing Reprogramming

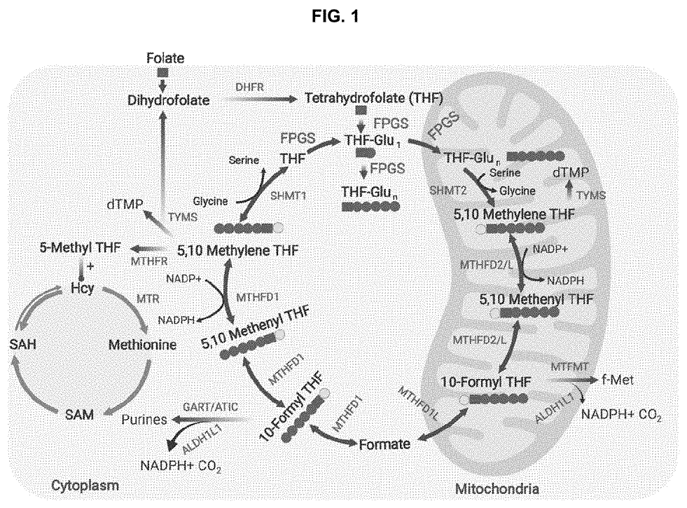

Tetrahydrofolate (THF) and its derivatives, collectively referred to as folates, constitute a group of cofactors critical for several major metabolic pathways in the cell. These cofactors participate in the addition and removal of one-carbon (C1) units in a set of reactions commonly referred to as C1 metabolism (1). The products of these C1 transfer reactions include purines, thymidylate, methionine, S-adenosyl methionine (SAM), and pantothenate (vitamin B5), all of which are crucial for normal cell function (2, 3). The partitioning of carbon units into various cellular outputs involves the following 4 major pathways: the folate cycle, the methionine cycle, the transsulphuration pathway, and the transmethylation metabolic pathways (4-6) (FIG. 1; Bio-Render, Toronto, ON, Canada). The transmethylation metabolic pathways closely interconnect choline methionine, and methyl-THF (7) and play a crucial role in the production of SAM (8).

Epigenetic marking by DNA and histone methylation depends on SAM (9). These epigenetic marks are established during mammalian development (10, 11), and are essential for the maintenance of cell identity (12, 13). A large body of evidence suggests that either deficiency or excess of folate can modulate DNA methylation in a cell-, gene-, and site-specific manner, and imbalanced methylation can reinforce a plethora of health conditions ranging from cardiovascular disease to depression (14-19). Loss-of-function mutations in enzymes that are involved in the folate cycle, methionine cycle, transsulphuration pathway, and the transmethylation metabolic pathways can lead to growth defects both in animals and in humans, underscoring the role of C1 metabolism in modulating cell growth (20, 21). Various studies indicate that a steady pool of folate cofactors is essential for actively dividing cells and is required for normal growth and development (22-24). Although much progress has been made toward understanding the biochemistry of enzymes involved in folate metabolism (25), genetic evidence for the biologic roles of these enzymes is still limited.

One of the key enzymes in folate metabolism is folypolyglutamate synthetase (FPGS), which catalyzes ATP-dependent sequential conjugation of Glu residues to folate, forming folypolyglutamates. Polyglutamylation is essential for the retention of folates within cellular compartments because nonglutamylated or monoglutamylated folates can transport across the mitochondrial membrane in either direction (25). The polyglutamate chain lengths of the folates differ from 1 cell type to another and within different organelles of a given cell, but in most eukaryotic cells, the penta- and hexaglutamate forms predominate (1). In mammals, there is only 1gene for FPGS, but there are 2 isoforms that are independently required in the cytosol and in the mitochondrial matrix (25, 26). Mitochondria receive folates from the cytoplasm only in a reduced, monoglutamylate form (FIG. 1), which is then polyglutamylated and charged with C1 units in situ (1). Folypolyglutamates cannot traverse mitochondrial membranes in either direction, so both mitochondrial and cytosolic isoforms of FPGS are required to maintain subcellular folate compartmentalization and function (25). Serine is oxidized in the mitochondria and is transferred to THF by serine hydroxylmethyltransferase (SHMT), resulting in glycine and 5,10-methylene-THF. A series of C1 reactions in mitochondria eventually produce formate, which flows to the cytoplasmic THF pool through the activity of mitochondrial methylene THF dehydrogenase (MTHFD) (27). The reductive incorporation of the formate into the cytosolic folate pool results in thymidine production (FIG. 1). Essentially, the cytosolic form of FPGS is required to synthesize purines and thymidine (28), and mitochondrial FPGS is required to produce glycine (29) in a mammalian cell.

In plants and cancer cells, mutation of FPGS changes the glutamylation status of the folates, and this alteration in polyglutamylated folates and associated compounds affects DNA methylation and releases chromatin silencing on a genome-wide scale (30-32). Polyglutamylated folates are better substrates for methylene THF reductase and methionine synthase, and both of these enzymes are involved in the generation of SAM (33, 34). In cancer cells, FPGS down-regulation by small interfering RNA reduces global DNA methylation and DNA methyltransferase (DNMT) activities (32, 35).

Although FPGS plays a central role in C1 metabolism, folate metabolism, and transmethylation pathways, it was unclear how an imbalance in these pathways caused by FPGS mutation would affect mammalian cell growth and differentiation. Therefore, a null mutant of FPGS was characterized in a mammalian cell, eliminating cytoplasmic and mitochondrial isoforms, and 4 splicing variants (36, 37). As disclosed herein, homozygous deletions of FPGS in the human embryonic kidney (HEK) 293T cell line were created using clustered regularly interspaced short palindromic repeats (CRISPR)/CRISPR-associated protein 9 (Cas9). The FPGS knockout)(FPGS^{ko}) cell lines are viable, displaying stem-cell markers in cell culture, but proliferate extremely slowly with a tendency toward cardiogenesis and neurogenesis.

The following examples are provided to better illustrate the claimed invention and the embodiments described herein, and are not to be interpreted as limiting the scope of the invention. To the extent that specific materials are mentioned, it is merely for purposes of illustration and is not intended to limit the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### Example 1: Elimination of Human Folypolyglutamate Synthetase Altered Programming and Plasticity of Somatic Cells

As demonstrated in the studies below, the elimination of both FPGS isoforms in 293T cells triggered epigenetic modifications, influenced gene expression, assisted cellular plasticity, and reduced cell proliferation. Moreover, the FPGS^{ko} cells are directed toward cardiac and neuronal lineages. A substantial reduction in global DNA methylation and noteworthy changes in gene expression related to C1 metabolism, cell division, DNA methylation, pluripotency, Glu metabolism, neurogenesis, and cardiogenesis were found. The expression levels of NANOG, octamer-binding transcription factor 4, and sex-determining region Y-box 2 levels were increased in the mutant, consistent with the transition to a stem cell-like state. Gene expression and metabolite data also indicate a major change in Glu and GABA metabolism. In the appropriate medium, FPGS^{ko} cells can differentiate to produce mainly cells with characteristics of either neural stem cells or cardiomyocytes.

### Materials and Methods

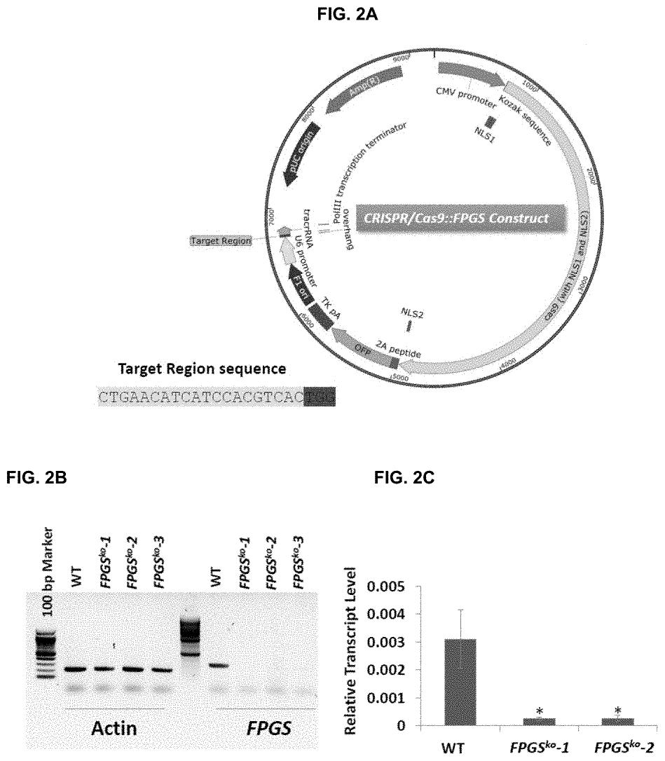

Cell lines and production of FPGS mutants: HEK cell line 293T was cultured in DMEM (Corning, Corning, N.Y., USA) supplemented with 10% fetal bovine serum (FBS; Thermo Fisher Scientific, Waltham, Mass., USA) and 1% GlutaMax (Thermo Fisher Scientific). The cell lines were cultured at 37° C. in a humidified 5% CO₂ incubator. Stable clonal cell lines were created by transfecting 293T cells with GeneArt CRISPR Nuclease Vector with orange fluorescent protein (OFP) reporter gene (Thermo Fisher Scientific) (38) (FIG. 2). For transfection, cells were seeded into a 6-well plate and transfected at 70% confluence using XFect (Takara, Kyoto, Japan) according to the manufacturer's protocol. Transfections were performed with 2 mg of a plasmid coexpressing Cas9, a chimeric single guide RNA (sgRNA), and OFP. At 24-36 hours post-transfection, cells were refreshed with 2 ml of growth medium and collected at 72 hours after transfection. Transfected positive clones were selected using single-cell sorting [BD FACSAria Cell Special Order Research Product Sorter (BD Biosciences, San Jose, Calif., USA)], and cells were collected in a 96-well plate for single-cell growth. Single-cell colonies were expanded in DMEM supplemented with 10% FBS (stem-cell quality; U.S. origin; Thermo Fisher Scientific), Minimum Essential Medium Nonessential Amino Acid (NEAA) solution (Thermo Fisher Scientific), and 1% Glutamax (Thermo Fisher Scientific), and evaluated by sequencing of genomic DNA.

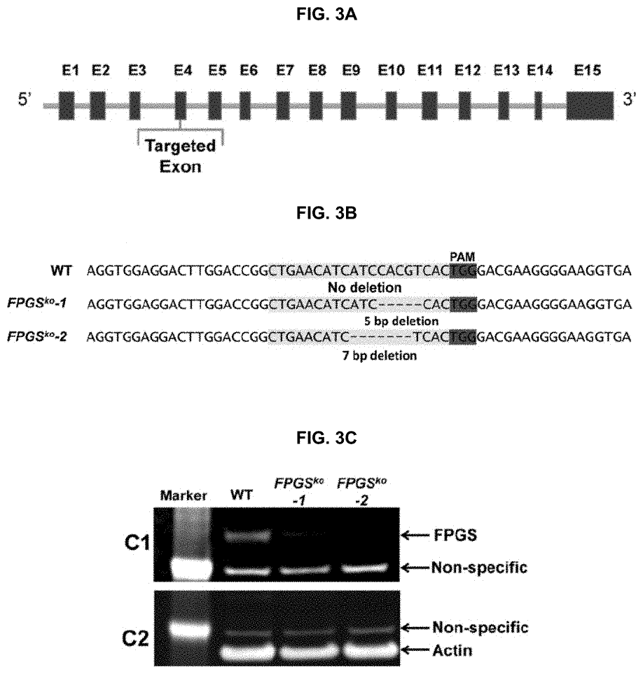

Plasmid and sgRNA design: The sgRNAs targeting the human FPGS gene were designed using Integrated DNA Technologies (Coralville, Iowa, USA) guide RNA (gRNA) design tools to minimize off-target, and the potency of these sgRNAs was also tested using the Basic Local Alignment Search Tool (BLAST; U.S. National Center for Biotechnology Information, Bethesda, Md., USA) analysis. The gRNAs were designed to target the conserved region of the FPGS and knockout function of both isoforms. The target sequences and plasmid construct map are shown in FIGS. 2 and 3. DNA oligos of sgRNAs were cloned using the GeneArt Seamless Cloning and Assembly Kit (Thermo Fisher Scientific) as per the manufacturer's protocol.

DNA and RNA isolation and analysis: Genomic DNA from putative clones was extracted using DNA-zol Reagent (Thermo Fisher Scientific) and a modified protocol of the previously published method in Ausubel et al. (39). For genotyping, PCR reactions were performed in duplicate with genomic DNA using High-Fidelity Taq DNA polymerase (Thermo Fisher Scientific) according to the manufacturer's protocol. The target-specific primer sets used for PCR are listed in Table 1 and Table 2 below.

TABLE 1
List of primers for genotyping, transcript analysis,
and complementation assay

| | | | | | PCR |
|---|---|---|---|---|---|
| | | | | | product |
| Gene | | | Experimental | Gene | Size |
| name | Primer | Primers sequence (5′-3′) | Purpose | ID | (bp) |
| FPGS | GT-F | CACTGGTCTGCTGGCTGTC (SEQ ID | Genotyping | 2356 | 661 |
| | | NO: 5) | | | |
| FPGS | GT-R | CCAATATGGTAAGTGCTAACTGAATG | Genotyping | | |
| | | (SEQ ID NO: 6) | | | |
| FPGS | GT-F1 | GTGACCCTCAGACACAGTTGG (SEQ | Genotyping | 2356 | 346 |
| | | ID NO: 7) | | | |
| FPGS | GT-R1 | CTAAAGAATCCCGTCTTCAGGC (SEQ | Genotyping | | |
| | | ID NO: 8) | | | |
| FPGS | sqRT-F | CATGCTCAATACCCTGCAG (SEQ ID | Semi-qRT-PCR | 2356 | 331 |
| | | NO: 9) | | | |
| FPGS | sqRT-R | CTTGGTGAAGAGCTCAGGACT (SEQ | Semi-qRT-PCR | | |
| | | ID NO: 10) | | | |
| FPGS | qRT-F | TGGAGTACCAGGATGCCGT (SEQ ID | qRT-PCR | 2356 | 207 |
| | | NO: 11) | | | |
| FPGS | qRT-R | CACAGGTGGAGCCCTTCC (SEQ ID | qRT-PCR | | |
| | | NO: 12) | | | |
| FPGS | WF-F* | ATGTCGCGGGCGCGGAGC (SEQ ID | Complementation | 2356 | 1761* |
| | | NO: 13) | | | |
| FPGS | WF-R* | CTGGGACAGTGCGGGCTC (SEQ ID | Complementation | | |
| | | NO: 14) | | | |

*Primers FPGS-WF-F and WF-R were used to amplify whole reading frame of FPGS from 293T for complementation assay.

TABLE 2
List of primers and genes for validation of transcriptomics data

| | | | | | PCR |
|---|---|---|---|---|---|
| | | | | | product |
| Gene | | | Experimental | | Size |
| name | Primer | Primers sequence (5′-3′) | Purpose | Gene ID | (bp) |
| bActin | F | CTTCCTTCCTGGGCATG (SEQ | Gene | NM_001101 | 204 |
| | | ID NO: 15) | Expression | | |
| | R | GAGCAATGATCTTGATCTTCAT | Gene | NM_001101 | |
| | | TG (SEQ ID NO: 16) | Expression | | |
| GTSF1 | F | GTGCAGAAAGAATCATCCTGA | Gene | NM_144594 | 229 |
| | | TG (SEQ ID NO: 17) | Expression | | |
| | R | CCACAAATCTTTATCCCAGTCT | Gene | NM_144594 | |
| | | TC (SEQ ID NO: 18) | Expression | | |
| SLC7A11 | F | CTATTTGGAGCTTTGTCTTATG | Gene | NM_014331 | 258 |
| | | CTG (SEQ ID NO: 19) | Expression | | |
| | R | CACTACAGTTATGCCCACAGC | Gene | NM_014331 | |
| | | T (SEQ ID NO: 20) | Expression | | |
| ANOS1 | F | GCTTTTGTGAGCCTCTCTTCC | Gene | NM_000216 | 241 |
| | | (SEQ ID NO: 21) | Expression | | |
| | R | GGGACACCTTTGTACAGAGTC | Gene | NM_000216 | |
| | | TTG (SEQ ID NO: 22) | Expression | | |
| GABRB2 | F | AGTCAATATGGATTATACCTTG | Gene | NM_021911 | 241 |
| | | ACAAT (SEQ ID NO: 23) | Expression | | |
| | R | GGTTGTGATTCTGAGTCCATAA | Gene | NM_021911 | |
| | | AG (SEQ ID NO: 24) | Expression | | |
| ANKRD1 | F | GTAGAGGAACTGGTCACTGGA | Gene | NM_014391 | 197 |
| | | AA (SEQ ID NO: 25) | Expression | | |
| | R | TTGAGCTCTGCCTCTCGTT | Gene | NM _014391 | |
| | | (SEQ ID NO: 26) | Expression | | |
| DKK1 | F | CAACTACCAGCCGTACCCG | Gene | NM_012242 | 184 |
| | | (SEQ ID NO: 27) | Expression | | |
| | R | CACACATATTCCATTTTTGCAG | Gene | NM_012242 | |
| | | T (SEQ ID NO: 28) | Expression | | |
| MTHFD2 | F | GGCAGTTCGAAATGAAGCTGT | Gene | NM_006636 | 204 |
| | | TG (SEQ ID NO: 29) | Expression | | |
| | R | CTGTTGATTCCCACAACTGCA | Gene | NM_006636 | |
| | | G (SEQ ID NO: 30) | Expression | | |
| ALDH1L2 | F | CACTGGCCGGGTTTATTTC | Gene | NM_001034173 | 167 |
| | | (SEQ ID NO: 31) | Expression | | |
| | R | CTGCAGCCAAAGCCAGAG | Gene | NM_001034173 | |
| | | (SEQ ID NO: 32) | Expression | | |
| Nanog | F | CAGCTACAAACAGGTGAAGAC | Gene | NM_024865 | 164 |
| | | CT (SEQ ID NO: 33) | Expression | | |
| | R | GGTTCACCAGGCATCCCT | Gene | NM_024865 | |
| | | (SEQ ID NO: 34) | Expression | | |
| Sox2 | F | CATGAATGCCTTCATGGTGT | Gene | NM_003106 | 182 |
| | | (SEQ ID NO: 35) | Expression | | |
| | R | GTGCTCCTTCATGTGCAGC | Gene | NM_003106 | |
| | | (SEQ ID NO: 36) | Expression | | |
| Oct4 | F | CGGAGGAGTCCCAGGACAT | Gene | NM_002701 | 142 |
| | | (SEQ ID NO: 37) | Expression | | |
| | R | CTGAATACCTTCCCAAATAGAA | Gene | NM_002701 | |
| | | CC (SEQ ID NO: 38) | Expression | | |

The total RNA from putative clones was extracted using the miRNeasy Mini Kit (Qiagen, Germantown, Md., USA). cDNA synthesis from 293T RNA was performed with 1 mg of total RNA using SuperScript III Reverse Transcriptase and the High-Capacity cDNA Reverse Transcription Kit (Thermo Fisher Scientific) according to the manufacturer's protocol.

Cell energy phenotype analysis using the Seahorse XFe96 extracellular flux analyzer. Basal mitochondrial function and metabolic potential of FPGS^{ko}-1 and wild-type (WT) cells were measured using the Seahorse Bioscience XFe96 Cell Energy Phenotype Test (Agilent Technologies, Santa Clara, Calif., USA). This assay simultaneously measures the 2 major energy-producing pathways in live cells (mitochondrial respiration and glycolysis), allowing a rapid determination of energy phenotypes of cells and investigating metabolic potential of the cell. The experiments were performed according to the manufacturer's protocol. Briefly, cells were seeded in DMEM supplemented with 10% FBS in 96-well tissue culture plates at a density of 20,000 cells/well and allowed to adhere for 24 hours. Prior to the assay, the medium was changed to DMEM containing 10 mM glucose, 1 mM pyruvate, and 2 mM Gln (pH 7.4), and the cells were equilibrated for 30 minutes at 37° C. The oxygen consumption rate (OCR) and extracellular acidification rate (ECAR) were measured under basal conditions. All treatment conditions were analyzed with 6-8 wells/treatment and repeated at least twice. OCR and ECAR values were normalized to cell numbers.

Clariom S Human Array and gene expression analysis: RNA was isolated using the RNeasy Mini Kit (Qiagen) according to the manufacturer's instructions. Total RNA was assessed for the RNA quality verification and microarray hybridization. The Agilent 2100 Bioanalyzer (Agilent Technologies), a microfluidics-based platform, was used for sizing, quantification, and quality of RNA. The RNA integrity number score was generated on the Agilent software. For the microarray analysis, the RNA quality for all of the samples had an RNA integrity number score>7.

For microarray analysis, 3 biologic replicates were included for both control and FPGS mutant. For each array experiment, 500 ng of total RNA was used for labeling using the Clariom S Human Array (Thermo Fisher Scientific). Probe labeling, chip hybridization, and scanning were performed according to the manufacturer's instructions. A Probe Set (gene-exon) was considered expressed if 50% samples had detection above background (DABG) values below the DABG threshold (DABG<0.05).

To validate microarray results, quantitative 2-step RT-PCR was performed. One microgram of total RNA was reverse transcribed to first-strand cDNA with the Qiagen cDNA Synthesis Kit (Qiagen), and this cDNA was subsequently used as a template for quantitative PCR with gene-specific primers. The ubiquitous β-actin gene served as a control for constitutive gene expression. The quantitative RT-PCR (qRT-PCR) reactions were performed using Power Sybr Green PCR Master Mix (Thermo Fisher Scientific) on a CFX96 Touch Real-Time PCR Detection System (Bio-Rad, Hercules, Calif., USA). Relative expression levels (2^{-ΔCt}) were calculated according to the Livak and Schmittgen method (40). Expression levels of each gene were compared with the expression level of actin. Values are the means of 3 biologic and 3 technical replicates and the oligonucleotides used in the study are presented in Tables 1 and 2.

Global DNA methylation measurement: Global 5-methylcytosine (5-mC) levels were quantified using the MethylFlash Methylated DNA Quantification Kits (Epigentek, Farmingdale, N.Y., USA). The DNA concentration was determined using the Qubit Assay (Thermo Fisher Scientific) according to the manufacturer's protocol. Briefly, 100 ng of DNA was used for incubation with both capture and detection antibodies using MethylFlash Methylated DNA Quantification Kit (Colorimetric) from Epigentek. Subsequently, measurements of the absorbance of the sample at 450 nm in a microplate spectrophotometer (BioTek Instruments, Winooski, Vt., USA) were performed with the percentage of the whole genome 5-mC calculation according to manufacturer's instructions. Genomic methylation levels in study samples were expressed as percentage of 5-mC.

Western blotting: Cells were lysed with Mammalian Protein Extraction Reagent lysis buffer (78501; Thermo Fisher Scientific) containing 1 mM PMSF and 1× protease inhibitor cocktail (MilliporeSigma, Burlington, Mass., USA). Proteins (40 mg/well) were separated on 4-12% gradient Bis-Tris NuPage gels (Thermo Fisher Scientific) and blotted on methanol-activated PVDF membrane (Thermo Fisher Scientific) using 1× transfer buffer (LC3625; Thermo Fisher Scientific) according to the manufacturer's instructions. Subsequently, blocking was performed using Li-Cor Biosciences (Lincoln, Nebr., USA) Odyssey blocking buffer (PBS; 927-40000). Thereafter, blocked membranes were incubated with a specific anti-human FPGS antibody (1:1000; AB184564; Abcam, Cambridge, Mass., USA) overnight at 4° C. in the same blocking buffer. The membrane was washed 3 times (5 minutes each wash) with 10 ml PBS with 0.05% Tween and 1× wash with 1×PBS on a shaker and incubated with anti-rabbit antibody dye 680RD (25-68071; Li-Cor Biosciences) for 90 minutes at room temperature. After incubation, the membrane was washed 3 times (5 minutes each wash) with 10 ml PBS with 0.05% Tween and 1× wash with 1×PBS on a shaker. Immunocomplexes were visualized with the Li-Cor Odyssey CLx in 700 channel (red). To visualize b-actin on the membrane, an anti-β-actin monoclonal antibody (012M4821; A1978; MilliporeSigma) and secondary antibody IR-Dye 800 (926-32210; Li-Cor Odyssey; Li-Cor Biosciences) were used.

Immunofluorescence analysis: The relative optimal image-based analysis of 2 key human pluripotent stem cells markers [octamer-binding transcription factor 4 (OCT4) and stage-specific embryonic antigen 4 (SSEA4)] was performed using the Pluripotent Stem Cell Immunocytochemistry Kit (Thermo Fisher Scientific) according to the manufacturer's instructions. Briefly, the cells were grown in 10% FBS DMEM supplemented with NEAAs and Glutamax in wells coated with 0.1% gelatin. For immunofluorescence localization, the cells were stained for SSEA4 and Oct4 and counterstained with DAPI using the kit (Thermo Fisher Scientific). The endogenous proteins were labeled using primary antibodies (anti-SSEA4 anti-mouse IgG3 and anti-OCT4 host-rabbit) followed by secondary antibodies conjugated to Alexa Fluor 488 goat anti-mouse IgG3 and Alexa Fluor 594 donkey anti-rabbit.

Chemical complementation assays of FPGS mutants: Cells were maintained as monolayers in DMEM with Gluta-Max (Corning) supplemented with 10% FBS at 37° C. in a 5% CO₂ atmosphere. To supplement the growth medium with amino acids, 1× and 2× doses of essential amino acids (Thermo Fisher Scientific) were added in the medium and the cells were grown as previously described for 5 days. In addition to this, Iscove's modified Dulbecco's medium (IMDM) and DMEM with 1×NEAAs (Thermo Fisher Scientific) along with FBS and Glutamax were used as described earlier. For 5-formyl-THF (5-CHO-THF) supplementation experiments, 6S-5-formyl-5,6,7,8-tetrahydrofolic acid (calcium salt; natural calcium folinate) was purchased from Schircks Laboratories (Jona, Switzerland). The stock solution (10 mM) of 5-CHO-THF was made using tissue culture-grade Dulbecco's PBS buffer (Thermo Fisher Scientific), and 100 ml from the stock solution was applied to the 1 ml IMDM to achieve the desired (1 mM) working concentration. The cells were grown for 5 days in the medium supplemented with 5-CHO-THF, and comparative analysis for cell growth was performed with the cells growing with only solvent control (only Dulbecco's PBS without 5-CHO-THF) in DMEM. All the experiments were carried out in triplicate, and cell proliferation was measured using a Cellometer Auto T4 Counting Chamber (Nexcelom Bioscience, Lawrence, Mass., USA).

Genetic complementation of FPGS mutants: To confirm that FPGSk® phenotype was caused by the loss of FPGS function, the WT-FPGS coding region from 293T was cloned and genetic complementation assays were performed on FPGS^{ko}-1 and FPGS^{ko}-2 mutants. Cells were grown (293T) in DMEM supplemented with 10% FBS, total RNAs were isolated, and first-strand cDNA was reverse transcribed as previously described. The FPGS coding region was amplified using the Phusion High-Fidelity DNA Polymerase (New England Biolabs, Ipswich, Mass., USA) and gene-specific primers (Tables 1 and 2). The resulting amplified product was cloned into pENTR-D-TOPO according to the manufacturer's protocol (Thermo Fisher Scientific), and the fragment was subsequently cloned into the pDest47 vector using gateway cloning according to the manufacturer's instructions (Thermo Fisher Scientific). The resulting expression plasmid (pDest47-FPGS-GFP) containing a functional fusion FPGS [FPGS fused to green fluorescent protein (GFP)] was delivered to FPGS^{ko}-1 and FPGS^{ko}-2 mutants using Lipofectamine 2000 Transfection reagent (Thermo Fisher Scientific) according to the manufacturer's protocol. Transfected positive clones (transiently expressing FPGS-GFP) were selected 3 days post-transfection using single-cell sorting as we did for selection of the FPGS^{ko} mutants. Expression of the transgenic FPGS was confirmed by qRT-PCR.

Metabolomics by hydrophilic interaction liquid chromatography and GC-MS: The cell lines (FPGS^{ko} and control) were cultured as described earlier in 100-mm plates. Once they attained confluency, the cells were washed twice with PBS (MilliporeSigma) and detached with 0.25% Trypsin-EDTA at 37° C. for 2 minutes. Subsequently, the cells were collected in 15-ml Falcon tubes, pelleted, and washed 5 times with ice-cold PBS before they were counted in a single-cell suspension using a Cellometer (Nexcelom Bioscience). The experiment was conducted with 5 biologic and 5 technical replicates, and a total of 8 million cells were used in each replicate. Samples were analyzed by the West Coast Metabolomics Center at the University of California-Davis (Davis, CA, USA) for primary metabolites (GC-MS) and biogenic amines [hydrophilic interaction liquid chromatography (HILIC)] using standard operating procedures as described earlier (41, 42).

Glu assay: The relative free Glu concentration in FPGS^{ko} mutants was assessed using a Glutamate Assay Kit (Abcam) according to the manufacturer's instructions. Using the kit, the free Glu levels in the mutant were measured and compared with the control cells (293T). The amount of Glu was quantified by colorimetric analysis (spectrophotometry at optical density=450 nm) using a Tecan Microplate Reader (Männedorf, Switzerland).

Cardiac and neural differentiation cell-culture methods: To induce differentiation, FPGS^{ko} cells were grown in human basal differentiation medium containing 10% FBS (stem-cell quality; Thermo Fisher Scientific), 1% NEAAs (Thermo Fisher Scientific), 1% penicillin-streptomycin (Thermo Fisher Scientific), 0.05 mM 2-ME (MilliporeSigma), and 2 mM L-GlutaMax in IMDM (Thermo Fisher Scientific) in 6-well ultra-low attachment plates until they reach 40-50% confluency. Subsequently, cells with embryoid body (EB)-like morphology were plated onto 0.1% gelatin-coated 12-well plates and grown in Roswell Park Memorial Institute (RPMI) 1640 medium with GlutaMax (Thermo Fisher Scientific) and serum-free B27 supplement (Thermo Fisher Scientific) differentiation factors and incubated from cardiac lineage to functional cardiomyocytes.

For neural differentiation, FPGS^{ko} cells were initially grown onto 0.1% gelatin-coated 12-well plates in DMEM supplemented with 10% FBS (stem-cell quality; Thermo Fisher Scientific), 1% NEAA (Thermo Fisher Scientific), 1% penicillin-streptomycin (Thermo Fisher Scientific), and 1% GlutaMax (Thermo Fisher Scientific). To induce neural differentiation, the cells were dissociated and passaged onto laminin (20 mg/ml; MilliporeSigma)-coated plates and grown in neurobasal medium (Thermo Fisher Scientific) or RPMI 1640 medium (Thermo Fisher Scientific) for 10-15 days at 37° C. and 5% CO₂. The medium was supplemented with 1% serum-free B-27 (Thermo Fisher Scientific), 1% Gluta-Max (Thermo Fisher Scientific), 1% NEAA (Thermo Fisher Scientific), and 1% penicillin-streptomycin (Thermo Fisher Scientific).

Cell quantification, imaging, and statistical analysis: Cells were quantified using a Cellometer Auto T4 counting chamber after mixing in a 1:1 ratio with Trypan blue (MilliporeSigma) to exclude dead cells. The average of 2 separate counts was taken to calculate the cell numbers. All live cell imaging was conducted using an EVOS FL Auto microscope (Thermo Fisher Scientific). All statistical analysis was performed using a paired Student's t test with Bonferroni correction or 1-sample Student's t test. Values of P<0.05 were considered significant.

### Results

Generation of FPGS depleted)(FPGS^{ko} human cells. In mammalian cells, the single gene for FPGS undergoes alternative splicing, resulting in 2 different isoforms, with the mitochondrial and cytosolic isoforms differing only in the N-terminal domain. To investigate the role of FPGS, deletions in both isoforms of FPGS in 293T cells (HEK cells) were generated using CRISPR/Cas9. The gRNAs used targeted conserved exons present in both isoforms. The sequence and targeting strategy are shown in FIGS. 2, 3A, and 3B. The targeting plasm id expressed OFP and fluorescence-activated cell sorting was used to isolate single-transfected cells, 15 of which slowly grew into small colonies over a period of about 60 days. PCR products of the targeted FPGS region from each putative clone were analyzed by sequencing. Two clones, FPGS^{ko}-1 and FPGS^{ko}-2, harbored 5- and 7-bp deletions, respectively, in the targeted region (FIG. 3B). The deletions were homozygous because all analyzed sequences showed the same deletion. The deletions in FPGS^{ko}-1 and FPGS^{ko}-2 cells both induce frame shifts, which result in premature stop codons. Western blots established that little or no FPGS protein was made in either mutant cell line (FIG. 3C); the mRNA was also rendered unstable because transcript levels were reduced over 10-fold (FIG. 2B).

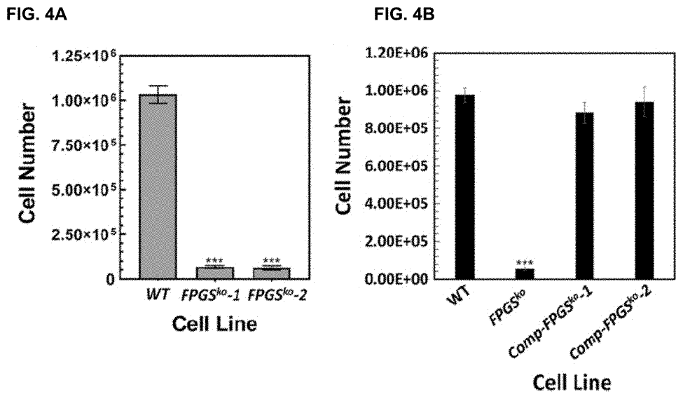

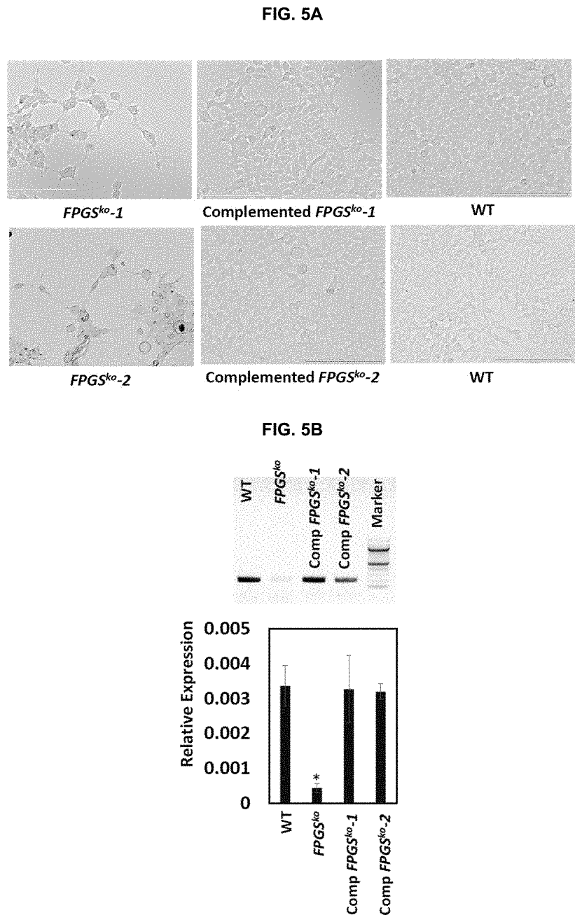

To confirm that the changes seen were not caused by off-target effects, complementation studies were performed. FPGS cDNA was prepared from WT (293T), cloned into an expression plasmid vector, forming pDest47-FPGS-GFP, which was used for transfection and for functional complementation. The hFPGS expression vector was transformed into FPGS^{ko}-1 and FPGS^{ko}-2, GFP-positive clones were selected using single-cell sorting, and expression of FPGS was verified using qRT-PCR. All clones complemented with FPGS showed relatively high levels of FPGS expression with cell growth and differentiation similar to that of WT 293T (FIGS. 4 and 5B). These findings indicate that the phenotypic alterations described herein arose from the disruption of FPGS. This conclusion is confirmed by medium supplementation results.

FPGS^{ko} cells show decreased cell proliferation. HEK cells (293T) exhibit rapid cell growth (43) in 10% FBS DMEM. However, of 15 putative clones for which growth was monitored (unpublished results), all clones exhibited extremely slow growth and a distinctive morphology that was different from the control cells (FIG. 4). Repeated passaging of the cells in DMEM supplemented with 10% FBS and NEAAs did not change the growth characteristics or morphologic features.

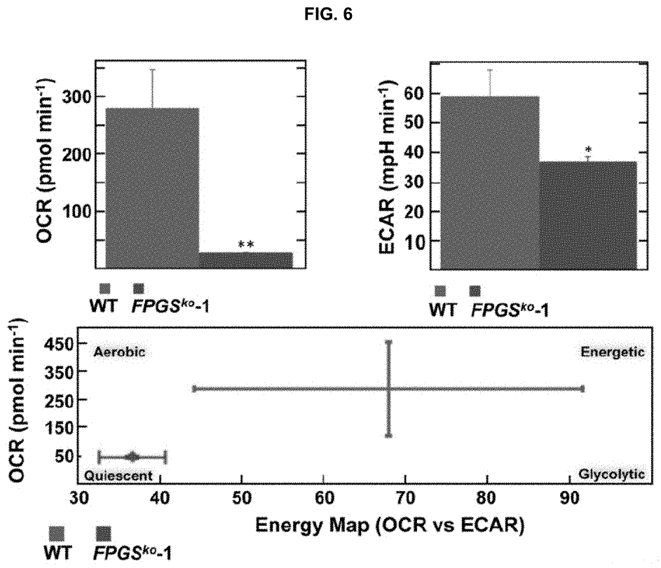

FPGS^{ko} mutants show reduced metabolic potential. To help understand the extremely slow growth of the mutants, cellular metabolism was examined by monitoring the OCR and ECAR of mutant and WT cells using the SeahorseXFe96. Both the mitochondrial respiration (OCR) and glycolytic activity (ECAR) of the FPGS^{ko}-1 cells were significantly decreased in comparison with control cells (FIG. 6), with a significant decrease of basal respiration, ATP production, and glycolytic capacity. These results indicate that the FPGS^{ko}-1 cells are in a quiescent-like state (FIG. 6), with oxidative phosphorylation being more affected than glycolysis.

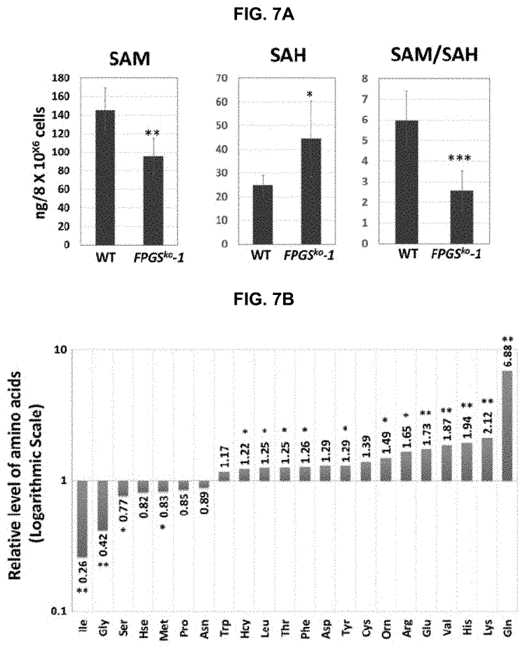

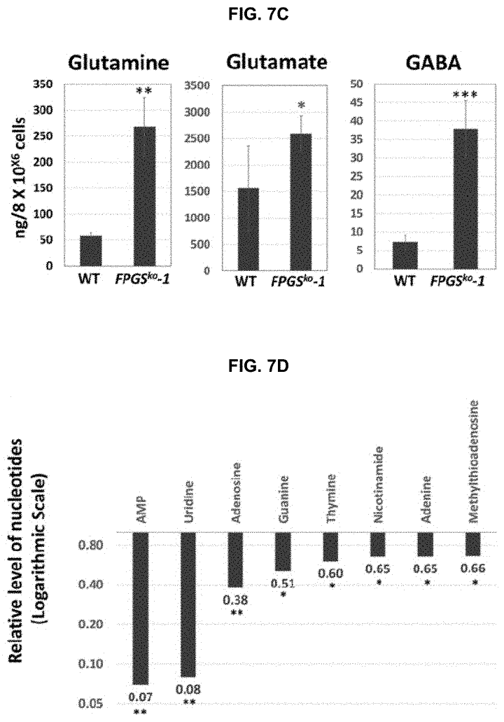

Metabolomic profiling of the FPGS^{ko} indicates a decreased methylation capacity and altered amino acid and nucleic acid profiles. HILIC- and GC-MS-based metabolomic approaches were carried out to understand the differential pattern of C1 and other primary metabolites in FPGS^{ko} cells as compared with WT cells. A total of 1488 (HILIC) and 503 (GC-MS) compounds were detected in FPGS^{ko} and 293T cells, of which 131 biogenic amines (by HILIC) and 165 primary metabolites (by GC-MS) could be assigned chemical structures and quantified based on spectral matching to authentic compounds. Considering the main focus of the study, the analysis was restricted to some key C1 compounds and primary metabolites. Compared with the parental cell, the metabolites belonging to the C1 pathway were changed significantly in FPGS^{ko} cells. The ratio of SAM to S-adenosylhomocysteine (SAH) provides an indication of the cellular capacity to catalyze transmethylation reactions. FPGS^{ko} cells had a significant accumulation of SAH along with a reduction in SAM. The resulting low SAM/SAH ratio in FPGS^{ko} suggests a marked difference in the methylation capacity of the mutant (FIG. 7A).

The analysis also showed that some nucleotides and amino acids were depleted in FPGS^{ko} (FIG. 7). Amino acids that showed a statistically significant reduction in FPGS^{ko} compared with the WT included Ile, Gly, Ser, Hse, Met, Pro, and Asn (FIG. 7B). However, Gln, Lys, His, Val, and Glu were significantly increased in FPGSk® compared with 293T cells (FIG. 7B). Quantitative analysis of Gln, Glu, and GABA further confirmed a significant accumulation of these metabolites in FPGS^{ko} cells (FIG. 7C). In addition to this, statistically significant reductions in the levels of AMP, uridine, adenosine, guanine, thymine, adenine, and methylthioadenosine in the mutant were observed (FIG. 7D).

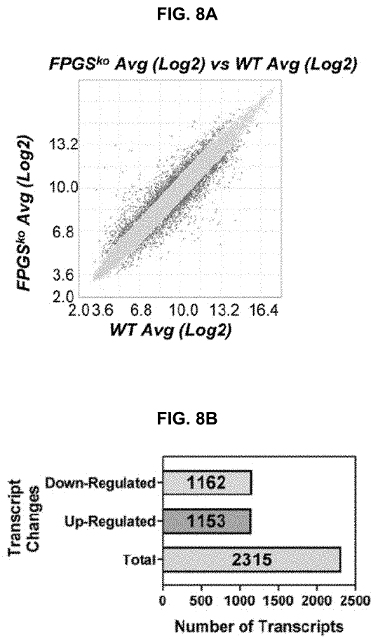

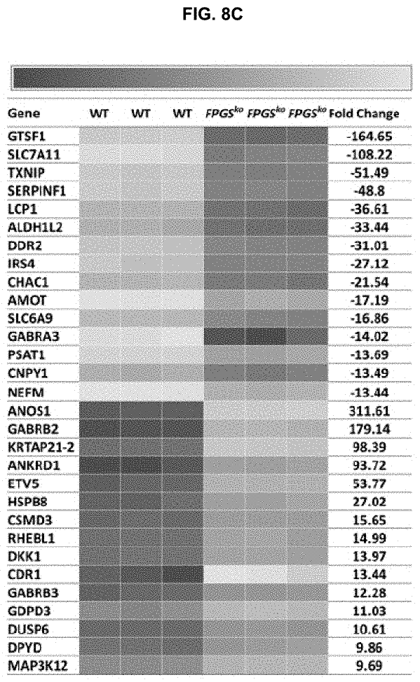

Microarray analysis identified 2315 differential genes in FPGS^{ko} cells. A transcriptional analysis of FPGS^{ko} was conducted using Clariom S Human Arrays. The cells were grown in DMEM (Corning) supplemented with 10% FBS (stem-cell quality; U.S. origin; Thermo Fisher Scientific) and 1% GlutaMax. These data revealed that 2315 genes were at least 2-fold differentially expressed between the FPGS^{ko} mutants and control cells. Among the differentially expressed genes, 1163 had higher expression in the FPGSk® mutant, whereas 1153 had a lower expression (FIG. 8). Major changes in expression were noticed for C1 metabolism, DNA methylation, cell cycle, cellular assembly and organization, Glu metabolism, developmental disorders, hereditary and neurologic disorders, DNA replication, and DNA repair genes.

Close examination of C1 metabolism-related genes showed that around 14 genes were significantly downregulated in the mutant, including thioredoxin-interacting protein (NM_006472), IGF-binding protein 2 (NM_000597), and cystathionine-b-synthase (NM_001178008). Among the genes directly involved in the folate biosynthesis pathway, expression of aldehyde dehydrogenase 1 family, member L2 (ALDH1L2; NM_001034173), MTHFD (NADP+dependent) 2 (NM_006636), and MTHFD (NADP+dependent) 1-like (NM_001242767) were significantly down-regulated in the mutant. Microarray data further validated that the FPGS (NM_001018078) was down-regulated in the FPGS^{ko}. In addition to this, expression of 36 genes associated with the methylation process and 26 genes related to DNA repair were affected.

These results were not unexpected based on the direct connection of FPGS to these pathways and process, but some unexpected results were observed in the up-regulated and down-regulated genes. Looking at the top 15 up-regulated genes in the transcript profiling of FPGS^{ko}, anosmin-1 (ANOS1) to be 311-fold higher in the mutant, followed by GABA A receptor, β-2 (179-fold), ankyrin repeat domain 1 (ANKRD1; 93-fold), E26 transformation specific (ETS) variant 5 (53-fold), heat shock 22 kDa protein 8 (27-fold), and expression of Dickkopf WNT-signaling pathway inhibitor 1 (DKK1) was 13-fold higher in the mutant as compared with the WT cells (FIG. 8). ANOS1 is a glycoprotein expressed in the brain and spinal cord (44). The GABA receptor is a multisubunit chloride channel that mediates the fastest inhibitory synaptic transmission in the CNS (45). Cardiac adriamycin-responsive protein or ANKRD1 is a cardiac ankyrin repeat protein that is highly expressed in cardiac and skeletal muscle (46). Interestingly, all these genes are associated with normal development and are associated with expansion and differentiation of neurons or cardiomyocytes.

Most of the genes down-regulated in FPGS^{ko} were associated with regulation of cell growth, differentiation, and metabolism. Some genes that manifested notably reduced expression in FPGS^{ko} as compared with the WT cells included gametocyte-specific factor 1 (GTSF1; 164-fold), solute carrier family 7 (SLC7; 108-fold), serpin peptidase inhibitor (48-fold), discoidin domain-containing receptor 2 (31-fold), insulin receptor substrate 4 (27-fold), and angiomotin (17-fold). Unbiased clustering analysis of the data suggests that FPGS elimination altered the expression of the genes related to cell differentiation, amino acid transport, angiogenesis, C1 metabolism, neurogenesis, and oxidative stress (FIG. 8).

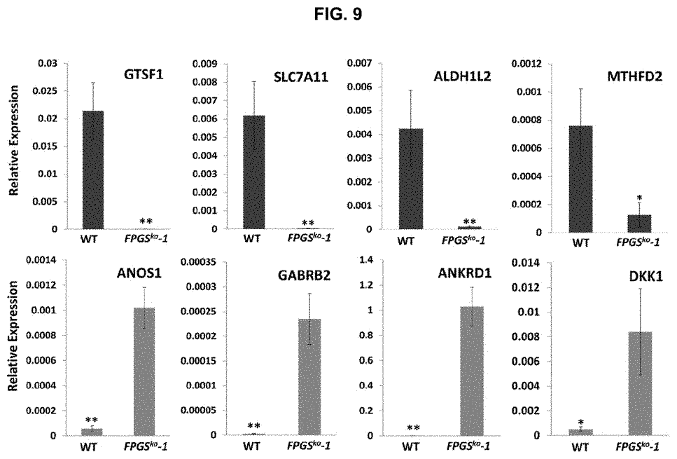

The microarray results for selected genes were validated by real-time quantitative PCR experiments using FPGS mutant and control cells. The transcript levels of GTSF1, SLC7A11, ALDH1L2, and MTHF were significantly repressed in the FPGS mutant, consistent with the microarray results (FIG. 9). Similarly, and consistent with microarray data, expression of ANOS1, GAB A receptor subunitb-2, ANKRD1, and DKK1 was significantly higher (FIG. 9) in the mutant when compared with 293T cells.

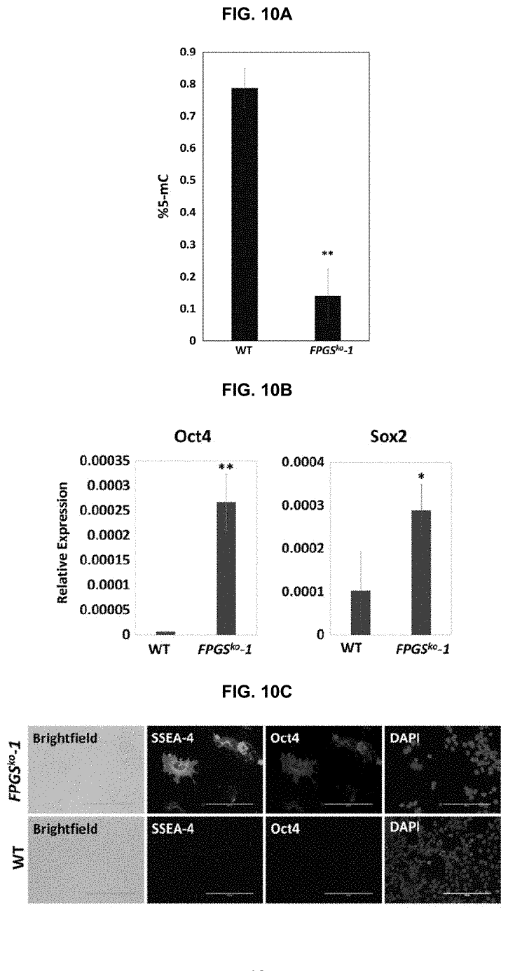

Global DNA methylation is reduced and FPGS^{ko} cells express pluripotent stem-cell markers. A global decrease in methylated DNA content has previously been observed after treatment with antifolates (15, 18, 19, 47, 48). Therefore, the global level of 5-mC was measured in FPGS^{ko} cells and a significant reduction in FPGS^{ko} cells was found (FIG. 10A).

Though slow growing, the morphologic features of FPGS^{ko} cells were similar to those of stem cells. Therefore, the markers for stem cells were examined in FPGS^{ko}-1 and FPGS^{ko}-2 cells grown on 0.1% gelatin-coated plates in DMEM supplemented with 10% FBS, Gln, and NEAAs. The expression of key pluripotency marker genes OCT4 and sex-determining region Y-box 2 (SOX2) were significantly higher in FPGS^{ko} clones when compared with parental lines (FIG. 10B). These findings were consistent with microarray data, and expression of around 25 pluripotency marker genes was significantly higher, including Sox2, Oct4, and Kruppel-like factor 4 in the mutant when compared with 293T cells. To authenticate these findings, 2 key pluripotency markers (OCT4 and SSEA4) were examined using immunochemical staining, and this revealed high levels of OCT4 and SSEA4 in FPGS^{ko} lines (FIG. 10C) as compared with control cells (FIG. 10C). Interestingly, a distinct staining pattern by OCT4 antibodies was observed in FPGS^{ko} lines, with staining observed in both cytosol and nucleus (FIG. 10B). Similarly, strong SSEA4 expression was observed in the mutant (FIGS. 10B and 10C). Together, these data indicate that defects in FPGS gene function cause somatic cells to lose cell identity and start expressing pluripotency genes.

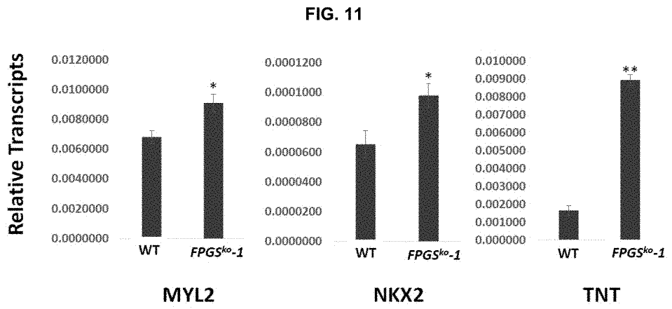

FPGS^{ko} cells can manifest hallmarks of cardiogenesis and neurogenesis. The 93-fold up-regulation of ANKRD1 [a protein that is highly expressed in stressed cardiac muscle (46)], 13-fold up-regulation of DKK1 [important in regulation of heart development, cardiac repair, and heart disease (49)], and 51-fold down-regulation of thioredoxin-interacting protein [controls cardiac hypertrophy through regulation of thioredoxin activity (50)], was suggestive of ailing cardiac progenitor cells (CPCs). To determine if FPGS^{ko} cells could form cardiomyocyte-like cells, an earlier reported cardiac differentiation protocol was adapted (51). Contractile EBs were noted at day 10. The contractile EBs in all groups peaked around day 14 and decreased after 17 days. The expression of cardiac-specific genes was also assessed in the FPGSk® cells by using RT-PCR. Expression of 3 key cardiomyocyte markers [Nkx2 (early cardiac transcriptional factor indicative of cardiac progenitor phenotype), myosin regulatory light chain 2 (a distinctly expressed protein in cardiac muscle), and cardiac troponin T (a muscle contractility regulatory protein indicative of a mature cardiac phenotype)]. All 3 were significantly up-regulated in the FPGS^{ko} cells grown in basal 10% FBS and DMEM (FIG. 11).

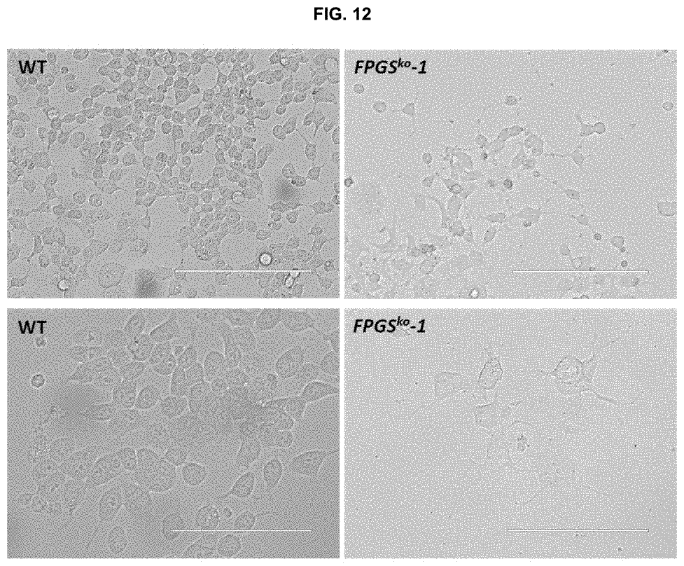

Neurogenesis was seen when FPGS^{ko} cells were maintained on laminin-coated plates and grown in neurobasal medium or RPMI 1640 basal medium with Glutamax-I and serum-free B27 supplement differentiation factors (FIG. 12). In a separate experiment, mutants formed neurons even if the cells were maintained on DMEM and not maintained on a specific differentiation medium. However, a consistent proportion and population of neurons were noted when they were grown in differentiation medium. Close microscopic examinations displayed many bipolar neurons in the neural population (FIG. 12).

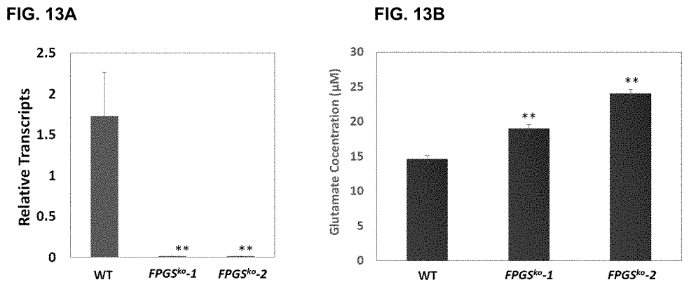

The neurotransmitters GABA and Glu are known to have a major role in survival, proliferation, and integration of newly formed neurons (52-54), and Glu can act as a positive regulator of neurogenesis (55). Gln also regulates CPC metabolism and proliferation in mammalian systems (56). The gene expression data pertaining to Gln-Glu-GABA metabolism were consistent with this notion, therefore, the free Glu concentration and expression of Glu-ammonia ligase were determined in the mutant. The expression of Glu-ammonia ligase was significantly low in the mutant, and free Glu concentration was significantly high in the FPGS^{ko} mutant (FIG. 13).

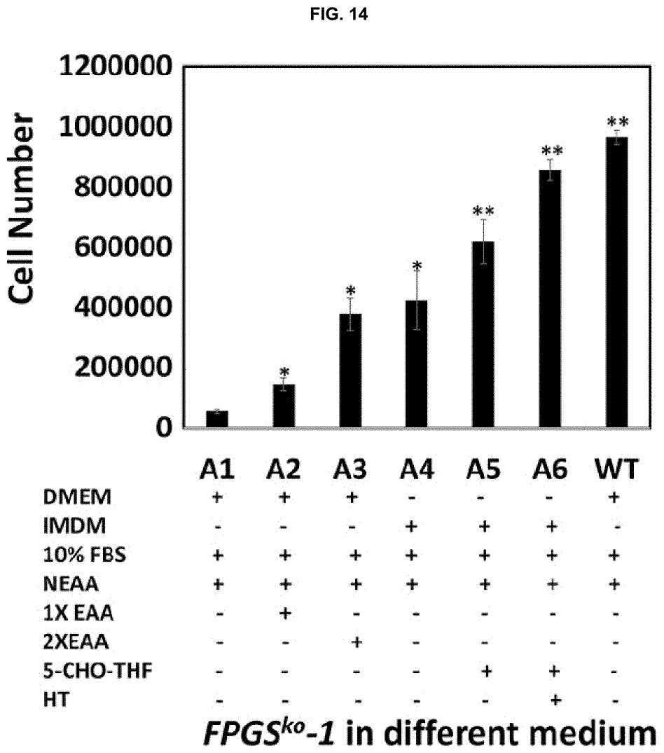

Nutrient supplementation rescues the FPGS^{ko} slow-growth phenotype. Whether supplementing the growth medium with various small molecules can rescue the FPGS^{ko} slow proliferation phenotype was tested. First, the cell-growth medium was spiked with additional essential amino acids (1×, 2×). Both FPGS mutants supplied with the additional essential amino acids showed increased cell proliferation (FIG. 14, A2-A3). Higher doses of amino acids (4×) in the medium were toxic (unpublished results). Because IMDM is recommended for embryonic stem-cell growth (57), FPGS^{ko} cells were grown in IMDM with 1× essential amino acids and NEAAs and an improvement in cell proliferation was noted (FIG. 14-A4). 5-CHO-THF and several amino acids are critical to the function of FPGS (58), so the FPGS^{ko} mutant was grown in IMDM supplemented with 1 mM 5-CHO-THF and 1×NEAAs and substantial increases in cell growth were observed (FIG. 14-A5). Finally, the FPGS^{ko} mutant was cultured in IMDM supplemented with 1 mM 5-CHO-THF, 1×NEAAs, hypoxanthine (10 mM), and thymidine (1.6 mM; Thermo Fisher Scientific). This modification showed almost full restoration of growth rate (FIG. 14-A6).

### Discussion

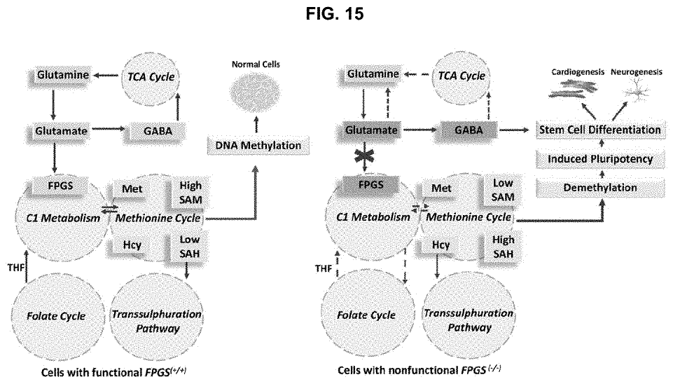

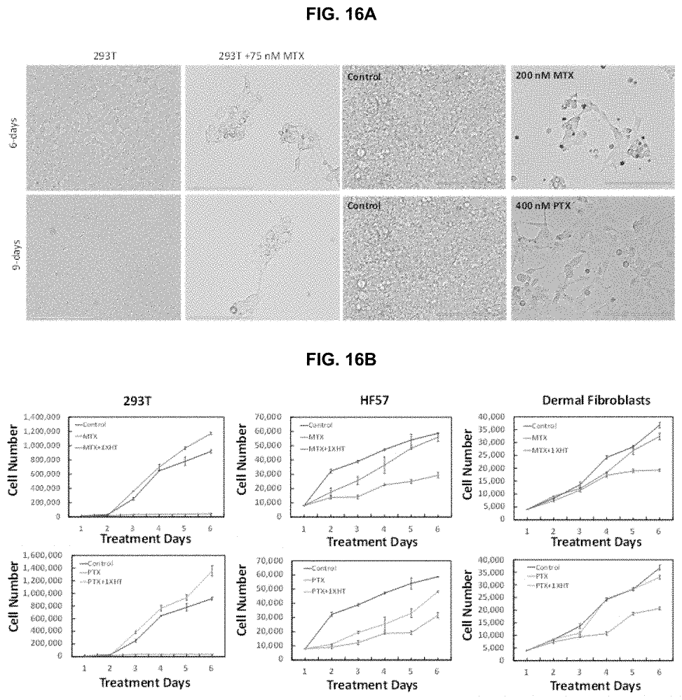

FPGS is a critical enzyme not only because it is required for intracellular folate homeostasis (30) but also because it links to the transmethylation pathway (FIGS. 1 and 15). The importance of FPGS for C1 metabolism in bacteria, yeast, and plant and mammalian cells (30, 59-62) is well established, but most studies in mammalian systems have been on cancer cells with the ultimate aim being cancer therapeutics (63-68). For example, Kim et al. (32) used RNA interference to knock down FPGS activity in breast cancer cells (32, 35) and found that FPGS modulation altered global DNA methylation and expression of several genes involved in important biologic pathways. Considering that the human FPGS gene produces 2 proteins by alternative translational initiation of exon 1 [Freemantle et al. (28)], and it has 4 splicing variants (36, 37), suppression of a specific isoform of FPGS is not enough to fully illustrate implications of FPGS disorder in a human cell. There is only 1 report of an FPGS-null mutant in mammalian cells (69); this investigation focused on formaldehyde toxicity and the diversion of endogenous formaldehyde into C1 metabolism, reporting that FPGS-null cells were not able to grow in unsupplemented growth medium. The role of FPGS in energy metabolism and cellular plasticity was not investigated. In this study, a conserved region of exon 4 of FPGS was targeted, which not only eliminates both isoforms of FPGS but also eliminates all splicing variants of FPGS. Additionally, around 34 single-nucleotide polymorphisms (SNPs) have been verified in FPGS that have altered the FPGS protein sequence (70). Among the 12 SNPs located in exon regions, none are known to be located in exon 4 (70). It is demonstrated herein that cells without FPGS are viable, although very slow growing in standard medium supplemented with 10% FBS, and undergo apparent reprogramming to a metabolic and transcriptional state with considerable resemblance to stem cells.

Somatic-cell reprogramming into stem cells using 4 transcription factors, Oct4, Sox2, Kruppel-like factor 4, and c-Myc or OCT4, SOX2, NANOG, and LIN28 is well-established (71-73), and suppression of the maintenance DNMT1 or treatment with the DNMT1 inhibitor 5-azacytidine can aid this conversion (74, 75). Folate in its various forms is essential for the conversion of homocysteine to SAM, which is the source of methyl groups for both DNA methylation and histone methylation. Folate deficiency and mutations in folate-dependent pathways are well known to affect mammalian development, even sometimes causing transgenerational effects, probably by affecting epigenetic inheritance (76). As an interesting example, a hypomorphic mutation in the mouse 5-methyl-THF-homocysteine methyltransferase reductase gene, which is required for activation of methionine synthase and thus the formation of SAM, results in congenital malformations that can persist through 5 generations (76). It is clear that a homeostatic balance among C1 metabolism, the methionine cycle, and the transmethylation metabolic pathways are required for normal cell function and development. Elimination of FPGS is expected to affect folate retention and function in both mitochondria and cytoplasm, and this is likely to have profound effects on C1 metabolism. Thus, the metabolism of FPGS^{ko} cells is greatly altered. A second finding is that FPGS^{ko} cells have features of stem cells.

Energy metabolism of stem cells is predominantly aerobic glycolysis (77). As demonstrated in the working example, the energy metabolism of FPGS^{ko} cells is also predominately glycolysis, though at a reduced level (FIG. 6). DNA methylation is reduced (FIG. 10) in FPGS^{ko} cells, which is consistent with an increased SAH/SAM ratio (FIG. 7), because SAH inhibits transmethylation reactions. This result is consistent with previous reports that perturbing folate and C1 metabolism affects global DNA methylation (15, 17, 32, 48, 76, 78, 79). Perhaps as a result of decreased DNA methylation, several key pluripotency genes such as OCT4, SOX2, and SSEA4 are expressed in FPGS^{ko} cells (FIGS. 10B and 10C). Additionally, as demonstrated in the working example, FPGS^{ko} cells will differentiate to either neuron-like cells or cardiomyocyte-like cells, depending on growth medium and conditions. Perhaps this is why the transcriptomic analysis of FPGS^{ko} cells showed, relative to parental 293T cells, greatly increased transcription of several neuronal and cardiomyocyte-specific genes. For example, the expression of ANOS1, GABA A receptor β-2, ANKRD1, and DKK1 was significantly higher in the mutant. ANOS1 and GABA play an important role in the CNS (45). Similarly, cardiac adriamycin-responsive protein or ANKRD1 is a rescue protein for cardiac muscle under stress conditions (46). At least some changes observed may be generally linked to FPGS reduction and not only to 293T being the parental cells, as a significant change in the expression of ANKRD1, DKK1, and SOX2 caused by FPGS modulation was also noticed by Kim et al. (32), who reduced FPGS levels in HCT116 colon and MDA-MB-435 breast cancer cells by small interfering RNA treatment.

It is not clear why differentiation is preferentially toward neurons or cardiomyocytes, but 1 possibility is a change in Gln and Glu metabolism. In FPGS^{ko} cells, increases in Gln (5-fold), Glu (1.7-fold), and GABA (5-fold) were observed. Glu is the key excitatory and GABA is the main inhibitory neurotransmitter in mammals (80, 81). The transcriptional analysis of FPGS^{ko} cells showed that the expression of 10 genes pertaining to Glu metabolism was significantly low. This includes glutathione-specific γ-glutamylcyclotransferase 1 (21-fold), asparagine synthetase (10-fold), and several neuronal and Glu transporters. In addition to this, expression of around 8 genes related to GABA receptors were significantly altered. Because Gln, Glu, and GABA are of special significance for neurons, the expression of neuron-related genes was checked. The expression of 20 genes connected to the brain and neurons were affected. Ras homolog enriched in brain-like 1, which was 15-fold higher in the FPGS mutant, has been associated with the neuronal development and hippocampal neurogenesis (82). In addition to this, 5-fold higher expression of brain acid-soluble protein (83) and 3-fold higher brain-derived neurotrophic factor were observed, which can stimulate neurogenesis in the cell culture (84).

Why do FPGS^{ko}-derived pluripotent cells preferentially differentiate into cardiomyocytes? There is a possible involvement of Gln, GABA, and C1 metabolism in CPC proliferation (56, 85-87). The GABA A receptor, which is abundant in the heart and brain, plays a significant role in cardiovascular regulation (88). GABA B receptors are also expressed and functional in mammalian cardiomyocytes (85). Interestingly, Salabei et al. (56) have shown that Gln is a primary regulator of CPC growth, differentiation, and survival.

In FPGS^{ko} cells, a distinct immunostaining of OCT4 was observed in both cytosol and the nucleus (FIG. 10C). Riekstina et al. (89) found that heart mesenchymal stem cells express OCT4, NANOG, SOX2, and SSEA4. Additionally, OCT4 expression is not always localized to the nucleus, but it is a nucleocytoplasmic shuttling protein (90), and expression of OCT4 mediates partial cardiomyocyte reprogramming of mesenchymal stromal cells (91). Altogether, it seems that the altered metabolic state of the FPGSk® cells predisposes them toward differentiation to cardiomyocytes and neurons. However, in normal, non-stem cell culture conditions, these cells are likely to be stressed, and this may explain the extremely high expression of ANKRD1, which is known to be expressed under stress conditions (46). Of interest, it has recently been reported that reduced cardiac hypertrophy and improved cardiac functions in mice is mediated by activation of serine and C1 metabolism (87).

### Example 2: Generating Multipotent Stem Cells by Treating Somatic Cells Transiently with MTX and/or PTX

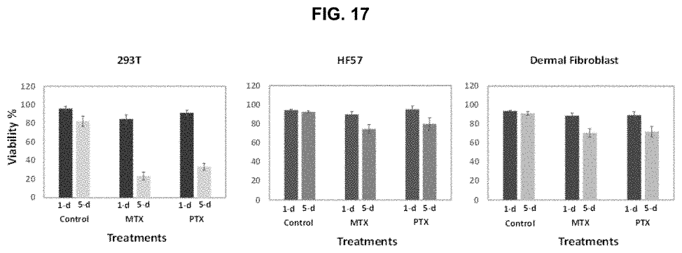

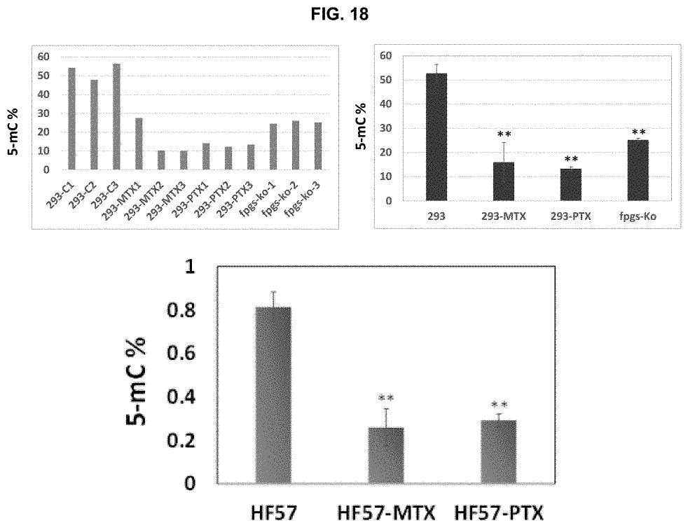

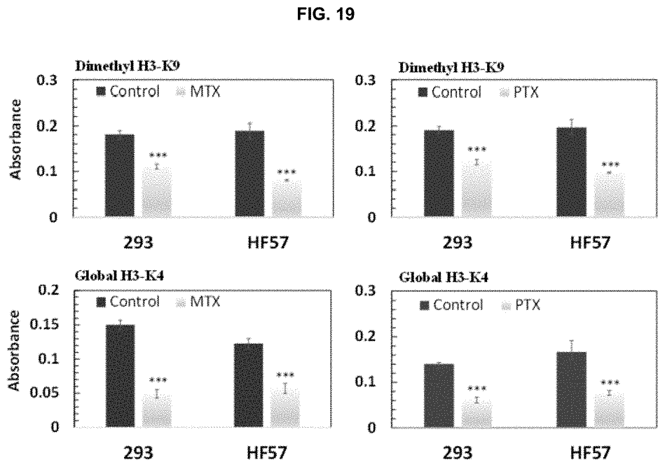

This example demonstrates generating multipotent stem cells (iMS cells) by treating somatic cells transiently with Methotrexate (MTX) and/or pemetrexed (PTX), a demethylating compound that is widely used in clinical practice. MTX and PTX are antifolates that inhibit enzymes (particularly thymidylate synthase and dihydrofolate reductase) involved in folate metabolism and purine and pyrimidine synthesis (100-103). Since these compounds are competitive inhibitors of dihydrofolate synthetase, these compounds were exogenously applied to the WT (HEK 293T and HF57) cells. As shown in FIGS. 16-17, application of MTX and PTX to the normal growing WT cells produced the phenotypes similar to the FPGS^{ko}. Several concentrations of MTX ranging from 75 nM to 500 nM were tested. The impact of MTX depended on the cell lines, exposure time, and the supplemental media. A concentration as low as 75 nM achieved similar results when a lower concentration of FBS was used, and the best results for 293T cells were obtained using MTX at a concentration ranging between 200 nM and 500 nM. Further, the pharmacological impacts of folate inhibitors on global DNA methylation were examined. 293T (control) cells were treated with 500 nM MTX and 1 μM PTX for at least 7 days before harvesting the cells and isolating the genomic DNA (gDNA). Equal amount of gDNA (100 ng) was analyzed with 5mC enzyme-linked immunosorbent assay (ELISA) (EpiGentek). The results indicate significantly lower levels of DNA methylation in the treated cells in comparison to controls (FIG. 18).

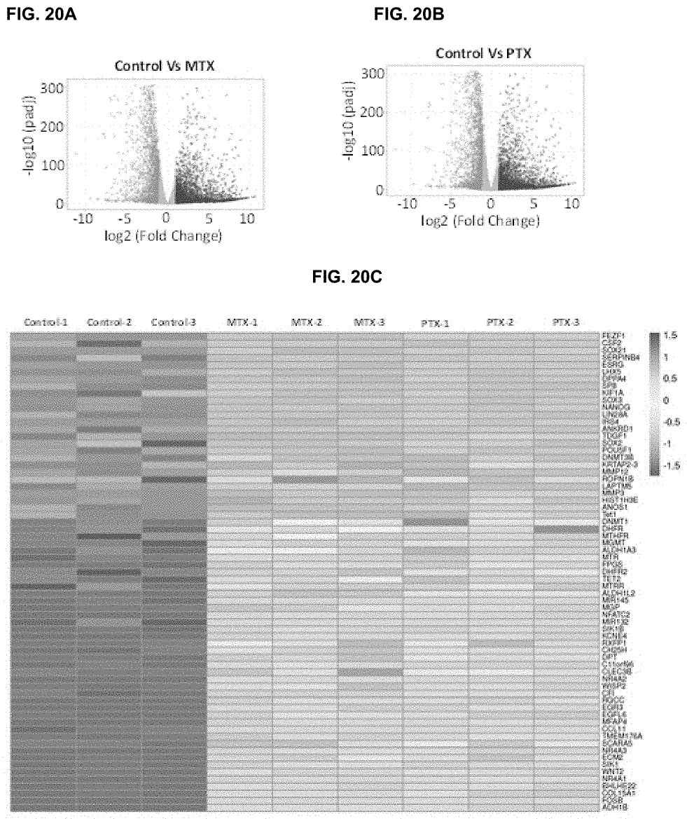

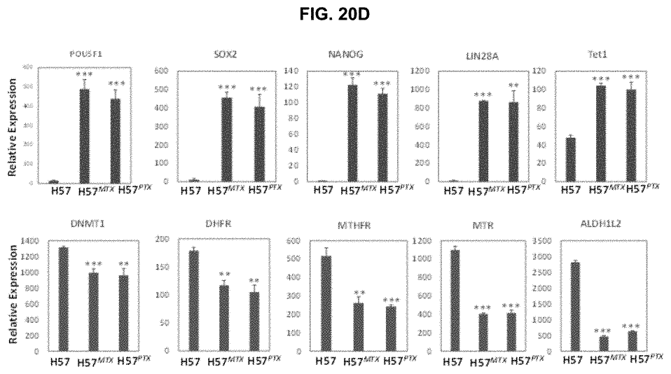

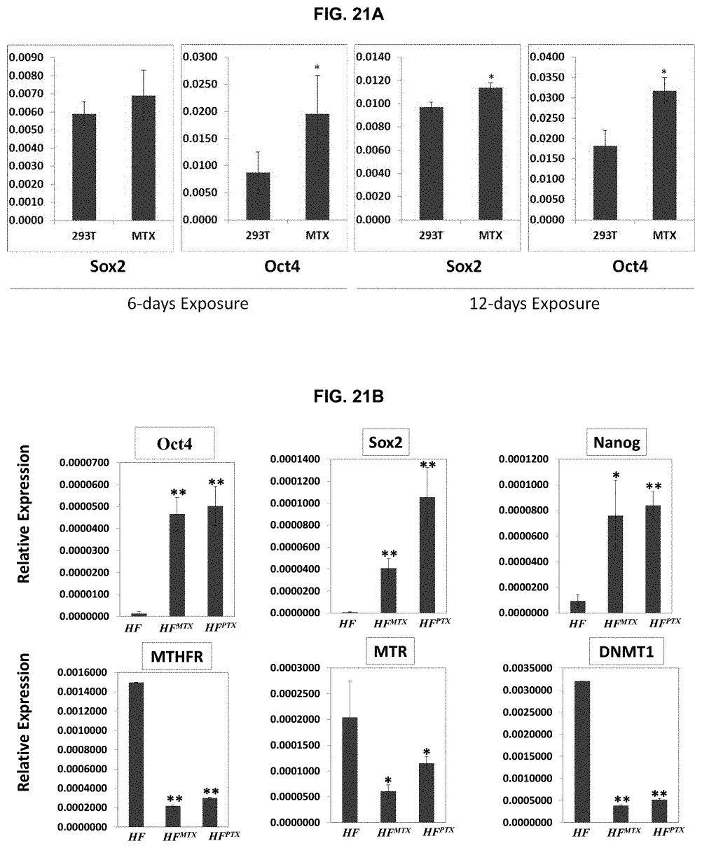

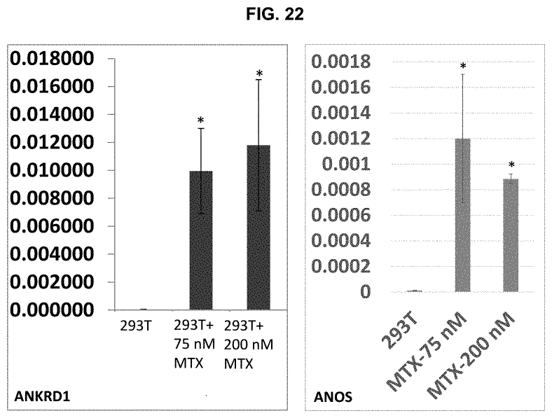

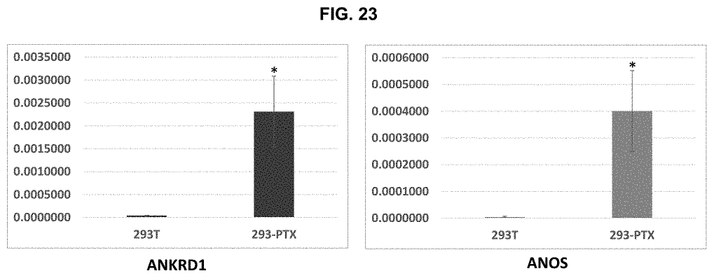

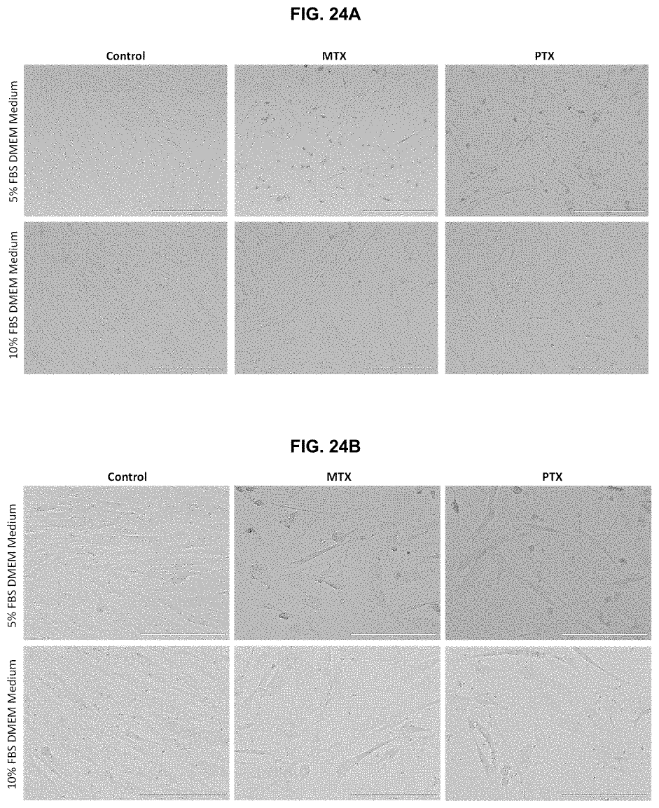

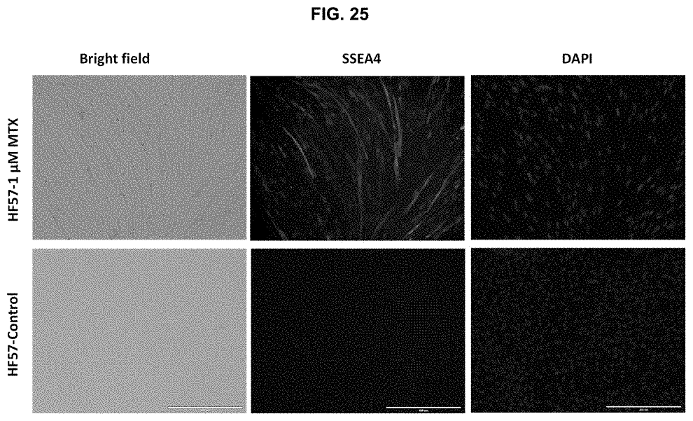

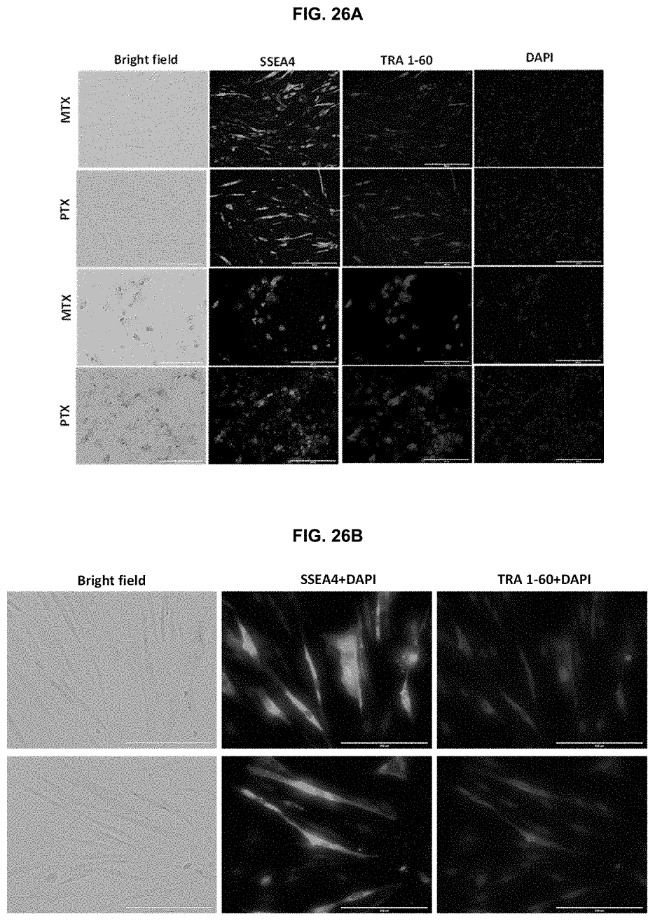

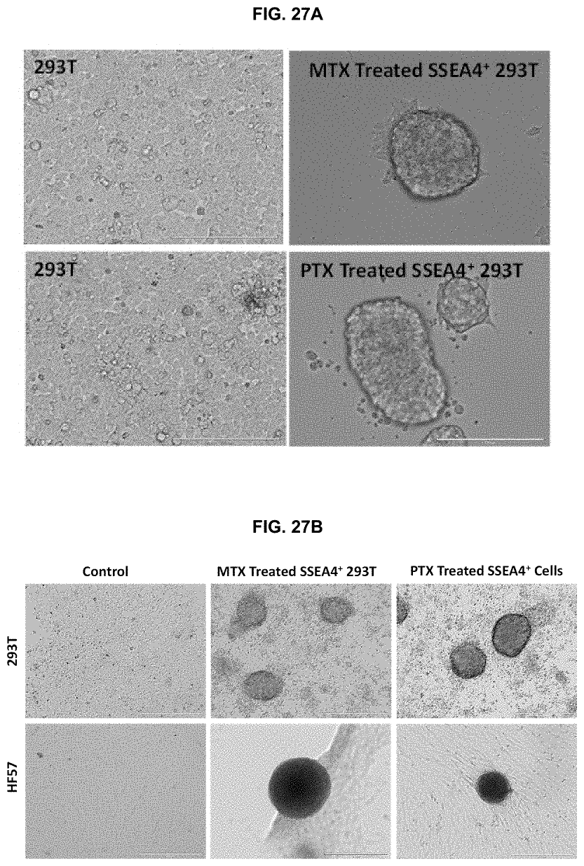

The quiescent nature of the MTX and PTX cells and the association of demethylation with the generation of induced pluripotent stem cells led to examining markers for stem cells formation. The 293T and HF57 cells were grown on 5% dialyzed FBS DMEM medium. DMEM without methionine, DMEM with 15 mg/L (half dose) and DMEM with 30 mg/L (regular DMEM) were used to grow the cells. Once the cells attained 70% confluency, HF57 were treated with 1-μM MTX or PTX and HEK293T were treated with 500 nM MTX or 1 μM PTX. The cells were treated with MTX or PTX for 7-days, and subsequently the medium was replaced with N2B27 medium (Thermo Fisher Scientific, USA). The cells were grown in N2B27 medium for 15 days which supported undifferentiated growth of human embryonic stem cells. Subsequently, these cells were maintained in MTeSR medium supplemented with sodium hypoxanthine and thymidine (HT) for 15 or more days. These conditions supported prolonged self-renewal of putative pluripotent cells, and they were able to form colonies and later embryoid bodies in vitro. Subsequently, the cells were assessed for markers linked to stem cells. Transcriptomics and qRT-PCR were performed to examine whether ES cell marker genes were expressed in these cells. Both MTX and PTX treated cells expressed the marker genes (FIGS. 20-21 and 25-26). The expression of key pluripotency marker genes OCT4 and SOX2 were significantly higher when compared to parental lines (FIGS. 20-21). FIG. 24 demonstrates that a better impact of MTX or PTX was observed at a lower FBS concentration, at 5%. To authenticate these findings, two key pluripotency markers (SSEA-4 and TRA 1-60) were examined using immunofluorescence. Immunochemical staining revealed high levels of SSEA-4 and TRA 1-60 in MTX treated HF57 lines (FIG. 26) as compared to control cells. Together, these data indicate that MTX and PTX treatment bring developmental changes in somatic cells and the treated cells lose cell identity and start expressing pluripotency genes.

A 3-germ layer immunocytochemistry kit (Thermo Fisher Scientific, USA) was used to assess the pluripotency of embryoid bodies derived from MTX and PTX treated cells. A comparative immunochemical analysis of embryoid bodies derived from MTX and PTX treated HF57 and HEK293T cells, human embryonic stem cells (H1), and iPSCs confirmed the presence of all three germ layers (FIG. 29). This confirms the presence of all three germ layers, and thus demonstrates the differentiation potency of reprogrammed cells treated with MTX or PTX.

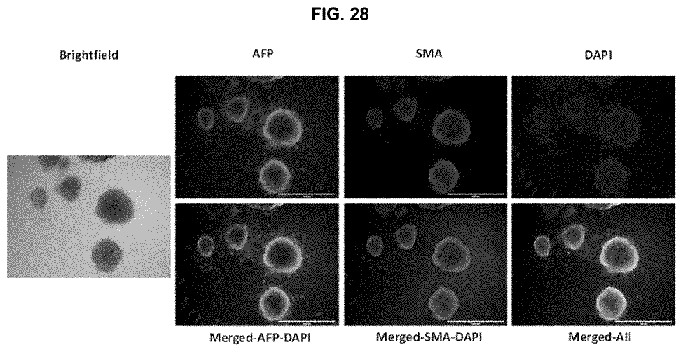

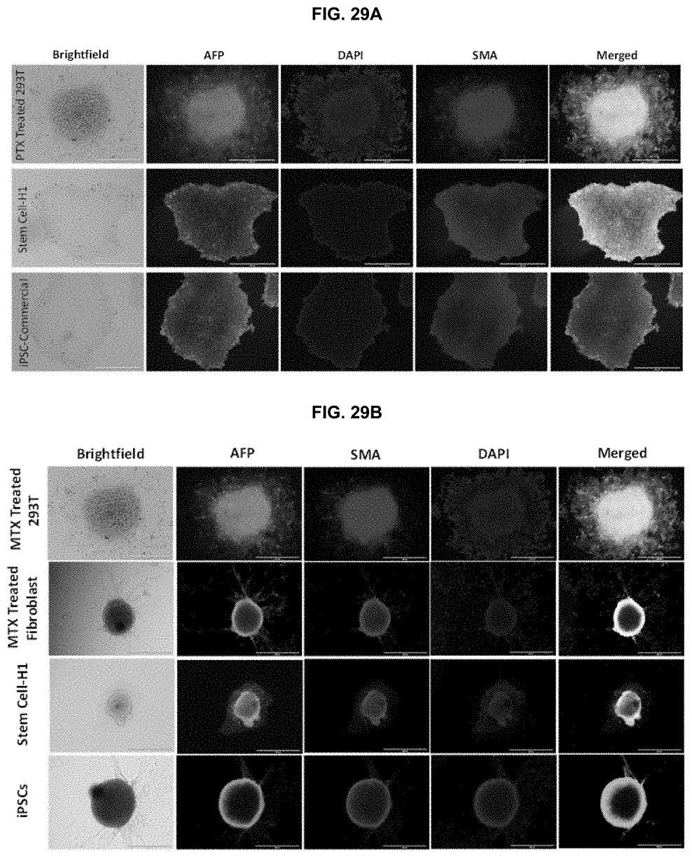

293T cells were treated with MTX and/or PTX for 10 or more days. SSEA4 positive cells were sorted using single cell sorter and maintained on mTeSR1 medium. After 10-days, cells started forming embryoid bodies indicating altered programing (Epigenetic and metabolic). Right panel shows embryoid bodies of MTX and/or PTX treated cells compared with embryoid bodies derived from human stem cell (FIG. 26). FIGS. 28-29 show the three-germ layer immunostaining of putative pluripotent stem cells derived from 293T cells treated with MTX and PTX.

### Example 3: Cardiac and Neural Differentiation Cell Culture Methods

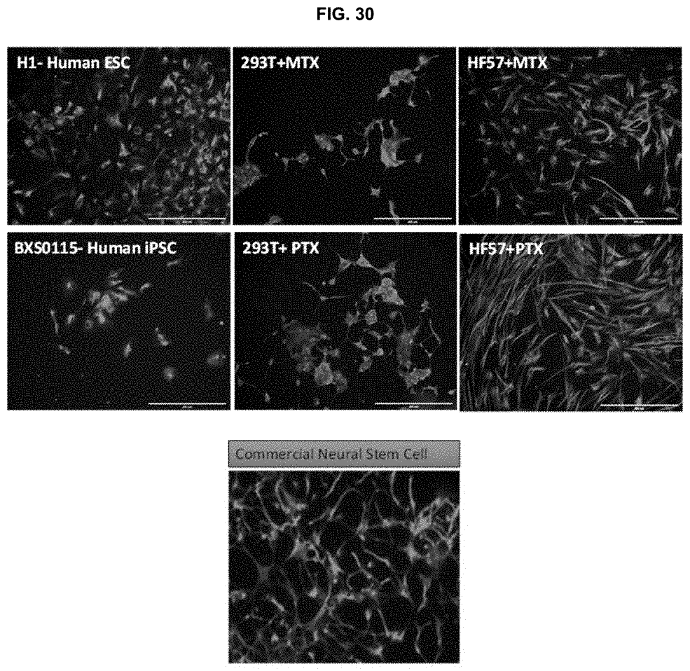

For neural differentiation, MTX/PTX treated, SSEA4 positive cells were initially grown onto Matrigel-coated 6-well plates in mTeSR1 medium. To induce neural differentiation, the cells were dissociated and passaged onto laminin (20 μg ml-1; Sigma-Aldrich, USA) coated plates and grown in Neurobasal medium (Thermo Fisher Scientific, USA) or RPMI1640 medium (Gibco, USA) for 10-15-days at 37° C./5% CO2. The medium was supplemented with 1% serum free B-27 (Gibco, USA), 1% Glutamax (Gibco, USA), 1% NEAA (Gibco, USA), and 1% penicillin/streptomycin (Gibco, USA). FIG. 30 demonstrates that MTX/PTX treated, SSEA4 positive cells can differentiate into neural cells in a suitable medium under suitable conditions.

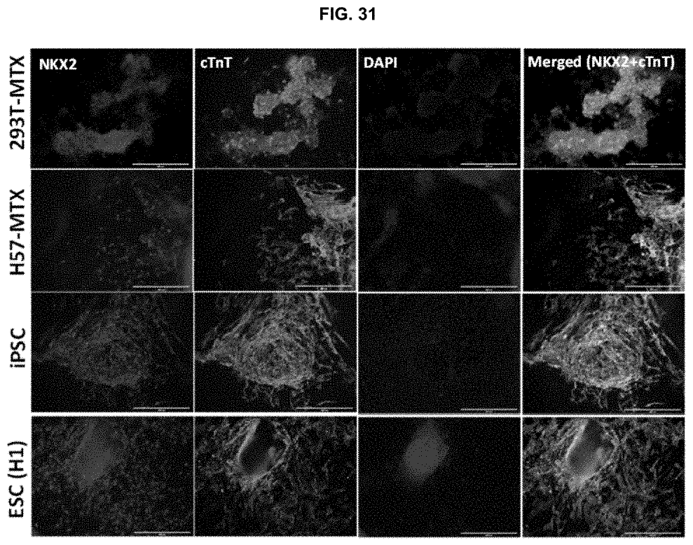

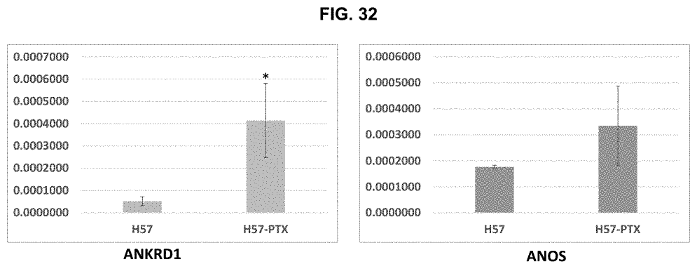

MTX/PTX treated and SSEA4 positive cells were maintained on Matrigel-coated 6-well plates in mTeSR1 medium. To induce differentiation, cells with EB-like morphology were dissociated with Accutase (Invitrogen) at 37° C. for 5 minutes and then single cell suspension was seeded onto a Matrigel-coated cell-culture dish at 100,000 cell/cm² in mTeSR1 supplemented with 5 μM ROCK inhibitor (Y-27632) for 24 hours. The cells then were cultured in mTeSR1 medium, which was changed daily. Next, 8 μl of 36 mM CHIR99021 were added into 24 ml RPMI/B27-insulin medium to make 12 μM CHIR99021 RPMI/B27-insulin medium and 2-ml of this were added to each well after the old mTeSR1 medium was removed. Exactly 24 hours after adding CHIR, the medium was replaced with 2 ml room temperature RPMI/B27-insulin. The plate was put back into the 37° C., 5% CO₂ incubator. 72 hours post addition of CHIR99021, 1 ml medium from each well was removed and the remaining 1-ml was supplemented with 1-ml RPMI/B27-insulin with 5 μM IWP2 (WNT pathway inhibitor). After 48 hours, the medium was replaced with fresh RPMI/B27-insulin and this was incubated at 37° C., 5% CO₂ incubator. After this, every 2-days, the old medium was removed from each well of the 12-well plate and 2 ml/well room temperature RPMI/B27 medium was added and incubated at 37° C., 5% CO₂. Spontaneous contraction should occur by day 14 and spontaneous beating can be maintained for several weeks. Correlative immunostaining of H1 (positive control), H57-untreated (negative control), H57 (MTX Treated), and 293 (MTX Treated) cell lines clearly show that the treated H57 and 293T cells had cardiac muscle cells in the differentiation medium. The expression of the key cardiomyocyte markers, Nkx2, which is an early cardiac transcriptional factor, indicative of cardiac progenitor phenotype, and cTNT, which is cardiac troponin T, a muscle contractility regulatory protein, indicative of a mature cardiac phenotype, were tested. Both were significantly up-regulated in the MTX or PTX treated cells grown in cardiomyocytes differentiation medium (FIG. 31).

### REFERENCES

1. Tibbetts, A. S., and Appling, D. R. (2010) Compartmentalization of mammalian folate-mediated one-carbon metabolism. Annu. Rev. Nutr. 30, 57-81
2. Anderson, O. S., Sant, K. E., and Dolinoy, D. C. (2012) Nutrition and epigenetics: an interplay of dietary methyl donors, one-carbon metabolism and DNA methylation. J. Nutr. Biochem. 23, 853-859
3. Locasale, J. W. (2013) Serine, glycine and one-carbon units: cancer metabolism in full circle. Nat. Rev. Cancer 13, 572-583
4. Shane, B., and Stokstad, E. L. (1985) Vitamin B12-folate interrelationships. Annu. Rev. Nutr. 5, 115-141
5. Stipanuk, M. H. (2004) Sulfur amino acid metabolism: pathways for production and removal of homocysteine and cysteine. Annu. Rev. Nutr. 24, 539-577
6. Stover, P. J. (2004) Physiology of folate and vitamin B12 in health and disease. Nutr. Rev. 62, S3-S12; discussion S13
7. Finkelstein, J. D. (1990)Methionine metabolism in mammals. J. Nutr. Biochem. 1, 228-237
8. Mato, J. M., Alvarez, L., Ortiz, P., and Pajares, M. A. (1997)S-adenosylmethionine synthesis: molecular mechanisms and clinical implications. Pharmacol. Ther. 73, 265-280
9. Kulis, M., and Esteller, M. (2010) DNA methylation and cancer. Adv. Genet. 70, 27-56
10. Riggs, A. D. (1975) X inactivation, differentiation, and DNA methylation. Cytogenet. Cell Genet. 14, 9-25
11. Chen, Z. X., and Riggs, A. D. (2011) DNA methylation and demethylation in mammals. J. Biol. Chem. 286, 18347-18353
12. Sheaffer, K. L., Kim, R., Aoki, R., Elliott, E. N., Schug, J., Burger, L., Schübeler, D., and Kaestner, K. H. (2014) DNA methylation is required for the control of stem cell differentiation in the small intestine. Genes Dev. 28, 652-664
13. Ziller, M. J., Ortega, J. A., Quinlan, K. A., Santos, D. P., Gu, H., Martin, E. J., Galonska, C., Pop, R., Maidl, S., Di Pardo, A., Huang, M., Meltzer, H. Y., Gnirke, A., Heckman, C. J., Meissner, A., and Kiskinis, E. (2018) Dissecting the functional consequences of de novo DNA methylation dynamics in human motor neuron differentiation and physiology. Cell Stem Cell 22, 559-574. e9
14. Balaghi, M., and Wagner, C. (1993) DNA methylation in folate deficiency: use of CpG methylase. Biochem. Biophys. Res. Commun. 193, 1184-1190
15. Friso, S., Choi, S.-W., Girelli, D., Mason, J. B., Dolnikowski, G. G., Bagley, P. J., Olivieri, O., Jacques, P. F., Rosenberg, I. H., Corrocher, R., and Selhub, J. (2002) A common mutation in the 5,10-methylenetetrahydrofolate reductase gene affects genomic DNA methylation through an interaction with folate status. Proc. Natl. Acad. Sci. USA 99, 5606-5611
16. Kim, J.-M., Hong, K., Lee, J. H., Lee, S., and Chang, N. (2009) Effect of folate deficiency on placental DNA methylation in hyperhomocysteinemic rats. J. Nutr. Biochem. 20, 172-176
17. Gonda, T. A., Kim, Y. I., Salas, M. C., Gamble, M. V., Shibata, W., Muthupalani, S., Sohn, K. J., Abrams, J. A., Fox, J. G., Wang, T. C., and Tycko, B. (2012) Folic acid increases global DNA methylation and reduces inflammation to prevent Helicobacter-associated gastric cancer in mice. Gastroenterology 142, 824-833.e7
18. Crider, K. S., Yang, T. P., Berry, R. J., and Bailey, L. B. (2012) Folate and DNA methylation: a review of molecular mechanisms and the evidence for folate's role. Adv. Nutr. 3, 21-38
19. Feng, H.-C., Lin, J.-Y., Hsu, S.-H., Lan, W.-Y., Kuo, C.-S., Tian, Y.-F., Sun, D.-P., and Huang, R. S. (2017) Low folate metabolic stress reprograms DNA methylation-activated sonic hedgehog signaling to mediate cancer stem cell-like signatures and invasive tumour stage-specific malignancy of human colorectal cancers. Int. J. Cancer 141, 2537-2550
20. Iskandar, B. J., Rizk, E., Meier, B., Hariharan, N., Bottiglieri, T., Finnell, R. H., Jarrard, D. F., Banerjee, R. V., Skene, J. H., Nelson, A., Patel, N., Gherasim, C., Simon, K., Cook, T. D., and Hogan, K. J. (2010) Folate regulation of axonal regeneration in the rodent central nervous system through DNA methylation. J. Clin. Invest. 120, 1603-1616
21. Wallingford, J. B., Niswander, L. A., Shaw, G. M., and Finnell, R. H. (2013) The continuing challenge of understanding, preventing, and treating neural tube defects. Science 339, 1222002
22. Mattson, M. P., and Shea, T. B. (2003) Folate and homocysteine metabolism in neural plasticity and neurodegenerative disorders. Trends Neurosci. 26, 137-146
23. Beaudin, A. E., and Stover, P. J. (2007) Folate-mediated one-carbon metabolism and neural tube defects: balancing genome synthesis and gene expression. Birth Defects Res. C Embryo Today 81, 183-203
24. Alata Jimenez, N., Torres P'erez, S. A., S'anchez-V'asquez, E., Fernandino, J. I., and Strobl-Mazzulla, P. H. (2018) Folate deficiency prevents neural crest fate by disturbing the epigenetic Sox2 repression on the dorsal neural tube. Dev. Biol. 444 (Suppl 1), S193-S201
25. Lawrence, S. A., Titus, S. A., Ferguson, J., Heineman, A. L., Taylor, S. M., and Moran, R. G. (2014) Mammalian mitochondrial and cytosolic folylpolyglutamate synthetase maintain the subcellular compartmentalization of folates. J. Biol. Chem. 289, 29386-29396
26. Lin, B. F., Huang, R. F., and Shane, B. (1993) Regulation of folate and one-carbon metabolism in mammalian cells. III. Role of mitochondrial folylpoly-gamma-glutamate synthetase. J. Biol. Chem. 268, 21674-21679
27. Luciano-Mateo, F., Hernandez-Aguilera, A., Cabre, N., Camps, J., Fernandez-Arroyo, S., Lopez-Miranda, J., Menendez, J. A., and Joven, J. (2017) Nutrients in energy and one-carbon metabolism: learning from metformin users. Nutrients 9, E121
28. Freemantle, S. J., Taylor, S. M., Krystal, G., and Moran, R. G. (1995) Upstream organization of and multiple transcripts from the human folylpoly-gamma-glutamate synthetase gene. J. Biol. Chem.270, 9579-9584
29. Taylor, R. T., and Hanna, M. L. (1982) Folate-dependent enzymes in cultured Chinese hamster ovary cells: impaired mitochondrial serine hydroxymethyltransferase activity in two additional glycine-auxotroph complementation classes. Arch. Biochem. Biophys. 217, 609-623
30. Srivastava, A. C., Ramos-Parra, P. A., Bedair, M., Robledo-Hernandez, A. L., Tang, Y., Sumner, L. W., Diaz de la Garza, R. I., and Blancaflor, E. B. (2011) The folylpolyglutamate synthetase plastidial isoform is required for postembryonic root development in Arabidopsis. Plant Physiol. 155, 1237-1251
31. Zhou, H.-R., Zhang, F.-F., Ma, Z.-Y., Huang, H.-W., Jiang, L., Cai, T., Zhu, J.-K., Zhang, C., and He, X.-J. (2013) Folate polyglutamylation is involved in chromatin silencing by maintaining global DNA methylation and histone H3K9 dimethylation in Arabidopsis. Plant Cell 25, 2545-2559
32. Kim, S. E., Hinoue, T., Kim, M. S., Sohn, K. J., Cho, R. C., Weisenberger, D. J., Laird, P. W., and Kim, Y. I. (2016) Effects of folylpolyglutamate synthase modulation on global and gene-specific DNA methylation and gene expression in human colon and breast cancer cells. J. Nutr. Biochem. 29, 27-35
33. McGuire, J. J., and Bertino, J. R. (1981) Enzymatic synthesis and function of folylpolyglutamates. Mol. Cell. Biochem. 38, 19-48
34. Moran, R. G. (1999) Roles of folylpoly-gamma-glutamate synthetase in therapeutics with tetrahydrofolate antimetabolites: an overview. Semin. Oncol. 26 (Suppl 6), 24-32
35. Kim, S.-E., Hinoue, T., Kim, M. S., Sohn, K.-J., Cho, R. C., Cole, P. D., Weisenberger, D. J., Laird, P. W., and Kim, Y.-I. (2015) g-Glutamyl hydrolase modulation significantly influences global and gene-specific DNA methylation and gene expression in human colon and breast cancer cells. Genes Nutr. 10, 444
36. Chen, L., Qi, H., Korenberg, J., Garrow, T. A., Choi, Y. J., and Shane, B. (1996) Purification and properties of human cytosolic folylpolygamma-glutamate synthetase and organization, localization, and differential splicing of its gene. J. Biol. Chem. 271, 13077-13087
37. Leclerc, G. J., and Barredo, J. C. (2001) Folylpoly-gamma-glutamate synthetase gene mRNA splice variants and protein expression in primary human leukemia cells, cell lines, and normal human tissues. Clin. Cancer Res. 7, 942-951
38. Liang, X., Potter, J., Kumar, S., Zou, Y., Quintanilla, R., Sridharan, M., Carte, J., Chen, W., Roark, N., Ranganathan, S., Ravinder, N., and Chesnut, J. D. (2015) Rapid and highly efficient mammalian cell engineering via Cas9 protein transfection. J. Biotechnol. 208, 44-53
39. Ausubel, F. M., Brent, R., Kingston, R. E., Moore, D. D., Seidman, J. G., Smith, J. A., and Struhl, K. (1989) Current Protocols in Molecular Biology, Vol. 1, John Wiley & Sons, Inc., Boston
40. Livak, K. J., and Schmittgen, T. D. (2001) Analysis of relative gene expression data using real-time quantitative PCR and the 2(-Delta C(T)) method. Methods 25, 402-408
41. Kind, T., Wohlgemuth, G., Lee, D. Y., Lu, Y., Palazoglu, M., Shahbaz, S., and Fiehn, O. (2009) FiehnLib: mass spectral and retention index libraries for metabolomics based on quadrupole and time-of-flight gas chromatography/mass spectrometry. Anal. Chem. 81, 10038-10048
42. Cajka, T., and Fiehn, O. (2016) Toward merging untargeted and targeted methods in mass spectrometry-based metabolomics and lipidomics. Anal. Chem. 88, 524-545
43. Hu, J., Han, J., Li, H., Zhang, X., Liu, L. L., Chen, F., and Zeng, B. (2018) Human embryonic kidney 293 cells: a vehicle for biopharmaceutical manufacturing, structural biology, and electrophysiology. Cells Tissues Organs (Print) 205, 1-8
44. Endo, Y., Ishiwata-Endo, H., and Yamada, K. M. (2012) Extracellular matrix protein anosmin promotes neural crest formation and regulates FGF, BMP, and WNT activities. Dev. Cell 23, 305-316
45. Srivastava, S., Cohen, J., Pevsner, J., Aradhya, S., McKnight, D., Butler, E., Johnston, M., and Fatemi, A. (2014) A novel variant in GABRB2 associated with intellectual disability and epilepsy. Am. J. Med. Genet. A. 164A, 2914-2921
46. Chu, W., Burns, D. K., Swerlick, R. A., and Presky, D. H. (1995) Identification and characterization of a novel cytokine-inducible nuclear protein from human endothelial cells. J. Biol. Chem. 270, 10236-10245
47. Wang, Y.-C., Shane, B., and Chiang, E. I. (2008) The effects of methotrexate and folate restriction on methionine metabolism in vitro and in vivo. Cancer Res. 68
48. Hanafy, S., Salem, T., El-Aziz, A., EL-Fiky, B., and Shokair, M. (2011) Influence of anticancer drugs on DNAmethylation in liver of female mice. Am. J. Mol. Biol. 1, 62-69
49. Pahnke, A., Conant, G., Huyer, L. D., Zhao, Y., Feric, N., and Radisic, M. (2016) The role of Wnt regulation in heart development, cardiac repair and disease: a tissue engineering perspective. Biochem. Biophys. Res. Commun. 473, 698-703
50. Yoshioka, J., Schulze, P. C., Cupesi, M., Sylvan, J. D., MacGillivray, C., Gannon, J., Huang, H., and Lee, R. T. (2004) Thioredoxin-interacting protein controls cardiac hypertrophy through regulation of thioredoxin activity. Circulation 109, 2581-2586
51. Wang, I. N., Wang, X., Ge, X., Anderson, J., Ho, M., Ashley, E., Liu, J., Butte, M. J., Yazawa, M., Dolmetsch, R. E., Quertermous, T., and Yang, P. C. (2012) Apelin enhances directed cardiac differentiation of mouse and human embryonic stem cells. PLoS One 7, e38328
52. Ge, S., Goh, E. L. K., Sailor, K. A., Kitabatake, Y., Ming, G. L., and Song, H. (2006) GABA regulates synaptic integration of newly generated neurons in the adult brain. Nature 439, 589-593
53. Vicini, S. (2008) The role of GABA and glutamate on adult neurogenesis. J. Physiol. 586, 3737-3738
54. Walls, A. B., Waagepetersen, H. S., Bak, L. K., Schousboe, A., and Sonnewald, U. (2015) The glutamine-glutamate/GABA cycle: function, regional differences in glutamate and GABA production and effects of interference with GABA metabolism. Neurochem. Res. 40, 402-409
55. Schlett, K. (2006) Glutamate as a modulator of embryonic and adult neurogenesis. Curr. Top. Med. Chem. 6, 949-960
56. Salabei, J. K., Lorkiewicz, P. K., Holden, C. R., Li, Q., Hong, K. U., Bolli, R., Bhatnagar, A., and Hill, B. G. (2015) Glutamine regulates cardiac progenitor cell metabolism and proliferation. Stem Cells 33, 2613-2627
57. Roxburgh, J., Metcalfe, A. D., and Martin, Y. H. (2016) The effect of medium selection on adipose-derived stem cell expansion and differentiation: implications for application in regenerative medicine. Cytotechnology 68, 957-967
58. Zhao, R., Babani, S., Gao, F., Liu, L., and Goldman, I. D. (2000) The mechanism of transport of the multi targeted antifolate (MTA) and its cross-resistance pattern in cells with markedly impaired transport of methotrexate. Clin. Cancer Res. 6, 3687-3695
59. Cody, V., Luft, J. R., Pangborn, W., Toy, J., and Bognar, A. L. (1992) Purification and crystallization of Lactobacillus casei folylpolyglutamate synthetase expressed in Escherichia coli. J. Mol. Biol. 224, 1179-1180
60. Atkinson, I. J., Nargang, F. E., and Cossins, E. A. (1998) Folylpolyglutamate synthesis in Neurospora crassa: primary structure of the folylpolyglutamate synthetase gene and elucidation of the met-6 mutation. Phytochemistry 49, 2221-2232
61. Andreassi J. L. II, and Moran, R. G. (2002) Mouse folylpoly-gammaglutamate synthetase isoforms respond differently to feedback inhibition by folylpolyglutamate cofactors. Biochemistry 41, 226-235
62. El Fadili, A., Kundig, C., and Ouellette, M. (2002) Characterization of the folylpolyglutamate synthetase gene and polyglutamylation of folates in the protozoan parasite Leishmania. Mol. Biochem. Parasitol. 124, 63-71
63. Moran, R. G., Colman, P. D., Harvison, P. J., and Kalman, T. I. (1988) Evaluation of pteroyl-S-alkylhomocysteine sulfoximines as inhibitors of mammalian folylpolyglutamate synthetase. Biochem. Pharmacol. 37, 1997-2003
64. McCloskey, D. E., McGuire, J. J., Russell, C. A., Rowan, B. G., Bertino, J. R., Pizzorno, G., and Mini, E. (1991) Decreased folylpolyglutamate synthetase activity as a mechanism of methotrexate resistance in CCRF-CEM human leukemia sublines. J. Biol. Chem. 266, 6181-6187
65. Rosowsky, A., Forsch, R. A., Null, A., and Moran, R. G. (1999) 5-deazafolate analogues with a rotationally restricted glutamate or ornithine side chain: synthesis and binding interaction with folylpolyglutamate synthetase. J. Med. Chem. 42, 3510-3519
66. Bienemann, K., Staege, M. S., Howe, S. J., Sena-Esteves, M., Hanenberg, H., and Kramm, C. M. (2013) Targeted expression of human folylpolyglutamate synthase for selective enhancement of methotrexate chemotherapy in osteosarcoma cells. Cancer Gene Ther. 20, 514-520
67. Huang, Z., Tong, H. F., Li, Y., Qian, J. C., Wang, J. X., Wang, Z., and Ruan, J. C. (2016) Effect of the polymorphism of folylpolyglutamate synthetase on treatment of high-dose methotrexate in pediatric patients with acute lymphocytic leukemia. Med. Sci. Monit. 22, 4967-4973
68. Wojtuszkiewicz, A., Raz, S., Stark, M., Assaraf, Y. G., Jansen, G., Peters, G. J., Sonneveld, E., Kaspers, G. J., and Cloos, J. (2016) Folylpolyglutamate synthetase splicing alterations in acute lymphoblastic leukemia are provoked by methotrexate and other chemotherapeutics and mediate chemoresistance. Int. J. Cancer 138, 1645-1656
69. Burgos-Barragan, G., Wit, N., Meiser, J., Dingler, F. A., Pietzke, M., Mulderrig, L., Pontel, L. B., Rosado, I. V., Brewer, T. F., Cordell, R. L., Monks, P. S., Chang, C. J., Vazquez, A., and Patel, K. J. (2017) Mammals divert endogenous genotoxic formaldehyde into one-carbon metabolism. Nature 548, 549-554; erratum: 612
70. Leil, T. A., Endo, C., Adjei, A. A., Dy, G. K., Salavaggione, O. E., Reid, J. R., Ames, M. M., and Adjei, A. A. (2007) Identification and characterization of genetic variation in the folylpolyglutamate synthase gene. Cancer Res. 67, 8772-8782
71. Takahashi, K., and Yamanaka, S. (2006) Induction of pluripotent stem cells from mouse embryonic and adult fibroblast cultures by defined factors. Cell 126, 663-676
72. Yu, J., Vodyanik, M. A., Smuga-Otto, K., Antosiewicz-Bourget, J., Frane, J. L., Tian, S., Nie, J., Jonsdottir, G. A., Ruotti, V., Stewart, R., Slukvin, I. I., and Thomson, J. A. (2007) Induced pluripotent stem cell lines derived from human somatic cells. Science 318, 1917-1920
73. Shi, Y., Desponts, C., Do, J. T., Hahm, H. S., Schöler, H. R., and Ding, S. (2008) Induction of pluripotent stem cells from mouse embryonic fibroblasts by Oct4 and Klf4 with small-molecule compounds. Cell Stem Cell 3, 568-574
74. Kim, K., Doi, A., Wen, B., Ng, K., Zhao, R., Cahan, P., Kim, J., Aryee, M. J., Ji, H., Ehrlich, L. I., Yabuuchi, A., Takeuchi, A., Cunniff, K. C., Hongguang, H., McKinney-Freeman, S., Naveiras, O., Yoon, T. J., Irizarry, R. A., Jung, N., Seita, J., Hanna, J., Murakami, P., Jaenisch, R., Weissleder, R., Orkin, S. H., Weissman, I. L., Feinberg, A. P., and Daley, G. Q. (2010) Epigenetic memory in induced pluripotent stem cells. Nature 467, 285-290
75. Pennarossa, G., Maffei, S., Campagnol, M., Tarantini, L., Gandolfi, F., and Brevini, T. A. (2013) Brief demethylation step allows the conversion of adult human skin fibroblasts into insulin-secreting cells. Proc. Natl. Acad. Sci. USA 110, 8948-8953
76. Padmanabhan, N., Jia, D., Geary-Joo, C., Wu, X., Ferguson-Smith, A. C., Fung, E., Bieda, M. C., Snyder, F. F., Gravel, R. A., Cross, J. C., and Watson, E. D. (2013) Mutation in folate metabolism causes epigenetic instability and transgenerational effects on development. Cell 155, 81-93
77. Ito, K., and Suda, T. (2014) Metabolic requirements for the maintenance of self-renewing stem cells. Nat. Rev. Mol. Cell Biol. 15, 243-256
78. Piyathilake, C. J., Johanning, G. L., Macaluso, M., Whiteside, M., Oelschlager, D. K., Heimburger, D. C., and Grizzle, W. E. (2000) Localized folate and vitaminB-12 deficiency in squamous cell lung cancer is associated with global DNA hypomethylation. Nutr. Cancer 37, 99-107
79. Li, Y., Feng, Q., Guo, M., Wang, Y., Jiang, Y., and Xing, J. (2018) Genome-wide survey reveals dynamic effects of folate supplement on DNA methylation and gene expression duringC2C12 differentiation. Physiol. Genomics 50, 158-168
80. Watkins, J. C., and Evans, R. H. (1981) Excitatory amino acid transmitters. Annu. Rev. Pharmacol. Toxicol. 21, 165-204
81. Hertz, L. (2013) The Glutamate-Glutamine (GABA) cycle: importance of late postnatal development and potential reciprocal interactions between biosynthesis and degradation. Front. Endocrinol. (Lausanne) 4, 59
82. Kang, E., Kim, J. Y., Liu, C. Y., Xiao, B., Chen, P. Y., Christian, K. M., Worley, P. F., Song, H., and Ming, G. L. (2015) Rheb1 mediates DISC1-dependent regulation of new neuron development in the adult hippocampus. Neurogenesis (Austin) 2, e1081715
83. Korshunova, I., Caroni, P., Kolkova, K., Berezin, V., Bock, E., and Walmod, P. S. (2008) Characterization of BASP1-mediated neurite outgrowth. J. Neurosci. Res. 86, 2201-2213
84. Huang, E. J., and Reichardt, L. F. (2001) Neurotrophins: roles in neuronal development and function. Annu. Rev. Neurosci. 24, 677-736
85. Lorente, P., Lacampagne, A., Pouzeratte, Y., Richards, S., Malitschek, B., Kuhn, R., Bettler, B., and Vassort, G. (2000) g-aminobutyric acid type B receptors are expressed and functional in mammalian cardiomyocytes. Proc. Natl. Acad. Sci. USA 97, 8664-8669
86. Tyagi, N., Lominadze, D., Gillespie, W., Moshal, K. S., Sen, U., Rosenberger, D. S., Steed, M., and Tyagi, S. C. (2007) Differential expression of gamma-aminobutyric acid receptor A (GABA(A)) and effects of homocysteine. Clin. Chem. Lab. Med. 45, 1777-1784
87. Padron-Barthe, L., Villalba-Orero, M., Gomez-Salinero, J. M., Acin-P'erez, R., Cogliati, S., Lopez-Olañeta, M., Ortiz-Sanchez, P., Bonzon-Kulichenko, E., Vazquez, J., Garcia-Pavia, P., Rosenthal, N., Enriquez, J. A., and Lara-Pezzi, E. (2018) Activation of serine one-carbon metabolism by calcineurin Ab1 reduces myocardial hypertrophy and improves ventricular function. J. Am. Coll. Cardiol. 71, 654-667
88. Chang, Y., Wang, R., Barot, S., and Weiss, D. S. (1996) Stoichiometry of a recombinant GABAA receptor. J. Neurosci. 16, 5415-5424
89. Riekstina, U., Cakstina, I., Parfejevs, V., Hoogduijn, M., Jankovskis, G., Muiznieks, I., Muceniece, R., and Ancans, J. (2009) Embryonic stem cell marker expression pattern in human mesenchymal stem cells derived from bone marrow, adipose tissue, heart and dermis. Stem Cell Rev Rep 5, 378-386
90. Oka, M., Moriyama, T., Asally, M., Kawakami, K., and Yoneda, Y. (2013) Differential role for transcription factor Oct4 nucleocytoplasmic dynamics in somatic cell reprogramming and self-renewal of embryonic stem cells. J. Biol. Chem. 288, 15085-15097
91. Yannarelli, G., Pacienza, N., Montanari, S., Santa-Cruz, D., Viswanathan, S., and Keating, A. (2017) OCT4 expression mediates partial cardiomyocyte reprogramming of mesenchymal stromal cells. PLoS One 12, e0189131
92. Lim, U., Peng, K., Shane, B., Stover, P. J., Litonjua, A. A., Weiss, S. T., Gaziano, J. M., Strawderman, R. L., Raiszadeh, F., Selhub, J., Tucker, K. L., and Cassano, P. A. (2005) Polymorphisms in cytoplasmic serine hydroxymethyltransferase and methylenetetrahydrofolate reductase affect the risk of cardiovascular disease inmen. J. Nutr. 135, 1989-1994
93. Heil, S. G., VanderPut, N. M., Waas, E. T., denHeijer, M., Trijbels, F. J., and Blom, H. J. (2001) Is mutated serine hydroxymethyltransferase (SHMT) involved in the etiology of neural tube defects? Mol. Genet. Metab. 73, 164-172
94. Ducker, G. S., and Rabinowitz, J. D. (2017) One-carbon metabolism in health and disease. Cell Metab. 25, 27-42
95. Soldner, F., Hockemeyer, D., Beard, C., Gao, Q., Bell, G. W., Cook, E. G., Hargus, G., Blak, A., Cooper, O., Mitalipova, M., Isacson, O., Jaenisch, R. (2009) Parkinson's disease patient-derived induced pluripotent stem cells free of viral reprogramming factors. Cell. 136, 964-977
96. Yu, J., Hu, K., Smuga-Otto, K., Tian, S., Stewart, R., Slukvin, II., Thomson, J. A. (2009) Human induced pluripotent stem cells free of vector and transgene sequences. Science. 324, 797-801
97. Warren, L., Manos, P. D., Ahfeldt, T., Loh, Y. H., Li, H., Lau, F., Ebina, W., Mandal, P. K., Smith, Z. D., Meissner, A., Daley, G. Q., Brack, A. S., Collins, J. J., Cowan, C., Schlaeger, T. M., Rossi, D. J. (2010) Highly efficient reprogramming to pluripotency and directed differentiation of human cells with synthetic modified mRNA. Cell Stem Cell. 7, 618-630
98. Zhou, H., Wu, S., Joo, J. Y., Zhu, S., Han, D. W., Lin, T., Trauger, S., Bien, G., Yao, S., Zhu, Y., Siuzdak, G., Scholer, H. R., Duan, L., Ding, S. (2009) Generation of induced pluripotent stem cells using recombinant proteins. Cell Stem Cell. 4, 381-384
99. Lyssiotis, C. A., Foreman, R. K., Staerk, J., Garcia, M., Mathur, D., Markoulaki, S., Hanna, J., Lairson, L. L., Charette, B. D., Bouchez, L. C., Bollong, M., Kunick, C., Brinker, A., Cho, C. Y., Schultz, P. G., Jaenisch, R. (2009) Reprogramming of murine fibroblasts to induced pluripotent stem cells with chemical complementation of Klf4. Proc Natl Acad Sci USA. 106(22), 8912-8917
100. Longley, D. B., D. P. Harkin, and P. G. Johnston. (2003) 5-fluorouracil: mechanisms of action and clinical strategies. Nature reviews. Cancer. 3, 330-338
101. Yamasaki, E., Y. Soma, Y. Kawa, and M. Mizoguchi. (2003) Methotrexate inhibits proliferation and regulation of the expression of intercellular adhesion molecule-1 and vascular cell adhesion molecule-1 by cultured human umbilical vein endothelial cells. The British journal of dermatology. 149, 30-38
102. Wu, M. F., Hsiao, Y. M., Huang, C. F., Huang, Y. H., Yang, W. J., Chan, H. W., Chang, J. T., and Ko, J. L. (2010) Genetic determinants of pemetrexed responsiveness and nonresponsiveness in non-small cell lung cancer cells. Journal of thoracic oncology: official publication of the International Association for the Study of Lung Cancer. 5, 1143-1151
103. Forster, V. J., McDonnell, A., Theobald, R., and McKay, J. A. (2017) Effect of methotrexate/vitamin B12 on DNA methylation as a potential factor in leukemia treatment-related neurotoxicity. Epigenomics, 9(9), 1205-1218

1. A method of reprogramming somatic cells into pluripotent stem cells, comprising:
contacting one or more somatic cells with one or more antifolate agents in vitro for a period of time sufficient to induce reprogramming; selecting cells expressing one or more stem cell markers; and
growing the selected cells to obtain the induced pluripotent stem cells (iPSCs).

2. The method of claim 1, wherein the somatic cell is a human somatic cell.

3. The method of claim 1, wherein the somatic cell is a fibroblast cell or a stromal cell.

4. The method of claim 1, further comprising contacting one or more somatic cells with methionine in vitro for a period of time sufficient to induce reprogramming.

5. The method of claim 1, wherein the method further comprising contacting the somatic cell with one or more of glutamine, glutamate, arginine, methionine, GABA, sodium hypoxanthine, and thymidine.

6. (canceled)

7. The method of claim 1, wherein the antifolate agent is an agent that inhibits one or more C1 metabolites, an agent that inhibits thymidylate synthase (TS), dihydrofolate reductase (DHFR), or both, or an agent that inhibits folypolyglutamate synthetase (FPGS).

8.-9. (canceled)

10. The method of claim 1, wherein the antifolate agent includes methotrexate (MTX), pemetrexed (PTX), aminopterin (MIT), raltitrexed, trimetrexate, piritrexim, edatrexate, and fluorouracil.

11. The method of claim 1, wherein the antifolate agent includes MTX, PTX, or both.

12. The method of claim 1, wherein the reprogrammed pluripotent stem cell expresses one or more of the markers including OCT4, SOX2, SSEA-4, Nanog, and TRA 1-60.

13. The method of claim 1, wherein the somatic cell is contacted with the antifolate agent for at least 1 day.

14. The method of claim 1, wherein the somatic cell is contacted with the antifolate agent for 1 day, 2 days, 3 days, 4 days, 5 days, 6 days, 7 days, 8 days, 9 days, 10 days, 11 days, 12 days, 13 days, 14 days, or 15 days.

15. A pluripotent stem cell obtained by reprogramming a somatic cell, the reprogramming comprises:
contacting the somatic cell with one or more antifolate agents in vitro for a period of time sufficient to induce reprogramming;
selecting cells expressing one or more stem cell markers; and
growing the selected cells to obtain the induced pluripotent stem cell, wherein the obtained pluripotent stem cell expresses one or more of the markers including OCT4, SOX2, SSEA-4, Nanog, and TRA 1-60.

16. The pluripotent stem cell of claim 15, wherein the reprogramming further comprises contacting the somatic cell with methionine in vitro for a period of time sufficient to induce reprogramming.

17.-18. (canceled)

19. The pluripotent stem cell of claim 15, wherein the somatic cell is a fibroblast cell or a stromal cell.

20. The pluripotent stem cell of claim 15, wherein the reprogramming further comprising contacting the somatic cell with one or more of glutamine, glutamate, arginine, methionine, GABA, sodium hypoxanthine, and thymidine.

21. The pluripotent stem cell of claim 15, wherein the antifolate agent is an agent that inhibits one or more C1 metabolites, an agent that inhibits thymidylate synthase (TS), dihydrofolate reductase (DHFR), or both, or an agent that inhibits folypolyglutamate synthetase (FPGS).

22.-23. (canceled)

24. The pluripotent stem cell of claim 15, wherein the antifolate agent includes methotrexate (MTX), pemetrexed (PTX), aminopterin (AMT), raltitrexed, trimetrexate, piritrexim, edatrexate, and fluorouracil.

25. The pluripotent stem cell of claim 15, wherein the antifolate agent includes MTX, PTX, or both.

26. The pluripotent stem cell of claim 15, wherein the somatic cell is contacted with the antifolate agent for at least 1 day.

27. The pluripotent stem cell of claim 15, wherein the somatic cell is contacted with the antifolate agent for 1 day, 2 days, 3 days, 4 days, 5 days, 6 days, 7 days, 8 days, 9 days, 10 days, 11 days, 12 days, 13 days, 14 days, or 15 days.
